(19) United States
(12) Patent Application Publication
(10) Pub. No.: US 2026/0108676 A1
(43) Pub. Date: Apr. 23, 2026

(54) JET EJECTION DEVICE

(71) Applicant: UNIVERSITEIT TWENTE, Enschede (NL)

(72) Inventors: David FERNANDEZ RIVAS, Enschede (NL); Jan Jelle SCHOPPINK, Enschede (NL); Keerthana MOHAN, Enschede (NL)

(73) Assignee: UNIVERSITEIT TWENTE, Enschede (NL)

(21) Appl. No.: 19/148,066

(22) PCT Filed: Jan. 19, 2024
(86) PCT No.: PCT/NL2024/050027

§ 371 Date: Jul. 15, 2025

(30) Foreign Application Priority Data
Jan. 20, 2023 (NL) 2034001

(51) Int. Cl. A61M 5/30

(52) U.S. Cl. CPC A61M 5/30; A61M 2005/3022; A61M 2205/368

(57) ABSTRACT

The invention provides a microfluidic device (1) for jet ejection, wherein the microfluidic device (1) comprises a hosting chamber (100) defined by a chamber wall (110), wherein the hosting chamber (100) is configured to host a liquid (10), wherein along a device axis of elongation (A_{D}) the hosting chamber (100) has a chamber length (L_{C}) defined by a first chamber end (101) and a second chamber end (102), wherein the first chamber end (101) comprises a first chamber opening (1011) for jet ejection from the hosting chamber (100); wherein the chamber wall (110) comprises a pattern (300) of a first surface material (111) and a second surface material (112), wherein the first surface material (111) has an equilibrium contact angle θ₁>90° for the liquid (10), and wherein the second surface material (112) has an equilibrium contact angle θ₂ for the liquid (10), wherein θ₁−θ₂≥20°; wherein the pattern (300) comprises a patch (200), wherein the patch has a patch boundary (205), and wherein (a) the patch (200) comprises one of the first surface material (111) and the second surface material (112), and wherein (b) at least 50% of the patch boundary (205) contacts the other of the first surface material (111) and the second surface material (112); wherein the chamber wall (110) has a wall surface area (S_{W}), wherein the patch (200) has a patch surface area (S_{P}), wherein 10⁻⁴≤S_{P}/S_{W}≤2*10⁻¹.

### FIELD OF THE INVENTION

The invention relates to a microfluidic device for jet ejection. Further, the present invention relates to a jet ejection system comprising the microfluidic device. The invention also relates to a method for ejecting a jet with the microfluidic device.

### BACKGROUND OF THE INVENTION

Microfluidic jet injection systems are known in the art. For instance, WO2021152476A1 describes a method for jet ejection of a fluid towards a substrate, comprising providing a nozzle filled inside with the fluid, positioning a fiber source of pulsed radiation inside the nozzle in direct contact with the fluid, the fiber source comprising an optical fiber, whereby the fluid is configured to absorb at least a part of the radiation, the method further comprising generating a bubble inside the nozzle by absorbing a pulse of the pulsed radiation in a first portion of the fluid, thereby vaporizing the fluid into the bubble, pushing a second portion of the fluid out of an opening at an extremity of the nozzle by an effect of an expansion of the bubble, whereby the extremity is directed to the substrate, thereby enabling the jet ejection of the second portion of fluid, and an intensity of the pulse being configured to be inferior to a radiation-induced threshold of a material comprised in the fiber source.

US20080186356A1 describes a nozzle plate having a nozzle hole formed therethrough, the nozzle hole being defined in the nozzle plate with an inner surface including a first liquid-philic portion, a liquid-phobic portion and a second liquid-philic portion that are arranged in this order from a side near the nozzle mouth, the first and second liquid-philic portions having liquid-philicity, the liquid-phobic portion having liquid-phoblicity.

US2022153028A1 describes a jet injection system comprising (i) a microfluidic device for jet ejection and (ii) a laser-based heating system, wherein: the microfluidic device comprises a hosting chamber defined by a chamber wall, the hosting chamber having a chamber height h_{c} selected from the range of 5-400 μm, a chamber width w_{c} selected from the range of 2h_{c}-10h_{c}, and a chamber length l_{c} defined by a first chamber end and a second chamber end, wherein the second chamber end comprises a first chamber opening for jet ejection from the hosting chamber, and wherein the hosting chamber is configured to host a liquid; the laser-based heating system is configured to provide laser radiation to one or more of the chamber wall and a liquid in the hosting chamber.

EP2388032A1 describes a device for creating at least one microfluidic jet, including at least one conduit section, open to an environment of the device at one end thereof. It further includes at least one arrangement for forming in at least one of the conduit sections a meniscus forming an interface between liquid in the conduit section and the environment, at a position within the conduit section. The meniscus is at least partially concave, seen looking into the conduit section through the end open to the environment. The device includes at least one holder for holding liquid, in fluid communication with at least one of the conduit sections. It also includes at least one device for delivering an energy pulse to liquid in at least one of the holders, configured to create a shock front propagating to the meniscus and causing at least a first stage of a jet to emerge from a central part of the meniscus.

### SUMMARY OF THE INVENTION

The present invention relates to a microjet injection system comprising a microfluidic device for generation of fluidic jets. More specifically, the system controls the jet in a way that does not solely depend on the geometry of the device or the input energy.

Microfluidic devices (also “microfluidic platforms” or “microfluidic systems”) comprise a broad range of devices related to the field of microfluidics. The field of microfluidics may deal with the behavior, control and manipulation of fluids, typically in small volumes, such as volumes on the order of μl, nl, pl, and fl. Microfluidic devices may be able to precisely control and manipulate fluids on a micrometer-size down to a sub-micrometer-size scale.

The use of microfluidic jets may be an alternative to conventional injection methods (such as by means of a needle). The term “jet” may also be referred to as “liquid jet” and “microfluidic jet”. Microfluidic jets may provide a method of injecting a liquid into a viscoelastic material, for example skin. Particularly, microfluidic jets may be a promising alternative to deliver drugs into skin.

Needle-injection has been used for the delivery of vaccination and medication. However, its invasive nature is a limiting factor. Jet injectors are a promising alternative to deliver drugs into the skin. Microfluidic devices for jet injection rely on accelerating a liquid jet to a velocity sufficient to penetrate the target viscoelastic material. Classical jet injectors may rely on a spring or a gas cartridge to create the jet. However, such devices may have limited control over the injection parameters such as volume and depth. Furthermore, they may require a nozzle to create jets thin enough to penetrate the target viscoelastic material.

Another crucial requirement for accurate and safe delivery of the liquid to the target viscoelastic material is jet stability, especially for applications such as delivery of medication through microjet injection. Due to fluidic instabilities (such as the Rayleigh-Plateau instability), the microfluidic jet may break up over time into smaller droplets with less inertia. Furthermore, the tail of the jet may have a reduced velocity or sway in a different direction (i.e., undesired angle of ejection of the jet). Both effects reduce the jet penetration power and could result in splash-back and/or incomplete delivery. This reduces the reproducibility and effectiveness of the microfluidic jet. Further, the splash-back may contaminate the jet ejection system and/or the microfluidic device.

Hence, it is an aspect of the invention to provide a microfluidic device for jet ejection, which preferably further at least partly obviates one or more of above-described drawbacks. The present invention may have as object to overcome or ameliorate at least one of the disadvantages of the prior art, or to provide a useful alternative.

Hence, in a first aspect the invention provides a microfluidic device for jet ejection. In embodiments, the microfluidic device may comprise a hosting chamber defined by a chamber wall. Especially, the hosting chamber may be configured to host a liquid. Along a device axis of elongation (A_{D}) the hosting chamber may in embodiments have a chamber length (L_{C}) defined by a first chamber end and a second chamber end. In embodiments, the first chamber end comprises a first chamber opening for jet ejection from the hosting chamber. In embodiments, the chamber wall may comprise a (surface) pattern of a first (repelling) surface material and a second (affinity) surface material. Especially, the first surface material may have an equilibrium contact angle θ₁>90° for the liquid. Further, the second surface material may especially have an equilibrium contact angle θ₂ for the liquid. In embodiments, θ₁−θ₂≥20°. In embodiments, the (surface) pattern may comprise a (surface) patch. Especially, the patch may be a two-dimensional shape defined on the surface of the chamber wall. In embodiments, the patch may have a patch boundary. Especially, the patch may comprise one of the first surface material and the second surface material. More especially, at least 50% of the patch boundary may contact the other of the first surface material and the second surface material. In embodiments, the chamber wall may have a (wall) surface area (S_{W}). Especially, the (surface) patch may have a (patch) surface area (S_{P}). In embodiments, 10⁻⁴≤S_{P}/S_{W}≤2*10⁻¹.

Hence, in specific embodiments, the invention provides a microfluidic device for jet ejection, wherein the microfluidic device comprises a hosting chamber defined by a chamber wall, wherein the hosting chamber is configured to host a liquid, wherein along a device axis of elongation (A_{D}) the hosting chamber has a chamber length (L_{C}) defined by a first chamber end and a second chamber end, wherein the first chamber end comprises a first chamber opening for jet ejection from the hosting chamber, wherein the chamber wall comprises a (surface) pattern of a first (repelling) surface material and a second (affinity) surface material, wherein the first surface material has an equilibrium contact angle θ₁>90° for the liquid, and wherein the second surface material has an equilibrium contact angle θ₂ for the liquid, wherein θ₁−θ₂≥20°, wherein the (surface) pattern comprises a (surface) patch, wherein the patch has a patch boundary, and wherein (a) the patch comprises one of the first surface material and the second surface material, and wherein (b) at least 50% of the patch boundary contacts the other of the first surface material and the second surface material, wherein the chamber wall has a (wall) surface area (S_{W}), wherein the (surface) patch has a (patch) surface area (S_{P}), wherein 10⁻⁴≤S_{P}/S_{W}≤2*10⁻¹.

Such a microfluidic device may create microfluidic jets with improved stability and reproducibility. Especially, the microfluidic device may comprise a heterogeneous surface chemistry for advanced control over the liquid and the microfluidic jet. Further, the chamber wall of the microfluidic device may have multiple different regions with different degrees of philicity (or “affinity”; for example hydrophilic and hydrophobic regions or lipophilic and lipophobic regions) inside the hosting chamber. Therefore, the attraction between the liquid and the chamber wall may be different at different positions inside the hosting chamber. This may alter the shape of the meniscus (the liquid-air interface) and provide more complex meniscus shapes. As the attraction of the liquid to a philic surface is larger, the capillary flow along a philic surface may generally be faster. Therefore, the meniscus will advance faster along the philic regions compared to phobic regions. This allows the creation of interface lines from philic to phobic regions where the meniscus is slowed (or its displacement is modified), allowing the creation of more reproducible filling levels. The control over the filling level is important for jet ejection, as the filling level may facilitate controlling the jet volume, and may further facilitate controlling the jet velocity, and thus (also) the injection depth. Further, the heterogeneous surface chemistry may (also) limit the tail to the same direction as the rest of the jet.

As mentioned above, the heterogenous surface chemistry of the chamber wall may be provided by the configuration of the patches (comprised by the pattern). Especially, the pattern may comprise two types of patches (i.e. patches that have different shapes, orientations, compositions, and material).

In embodiments, the patches may (generally) be elongated. In further embodiments, the patches may be (i) configured perpendicular (or nearly perpendicular) to the device axis of elongation (A_{D}), or (ii) configured parallel (or nearly parallel) to the device axis of elongation (A_{D}).

The patches configured perpendicular (or nearly perpendicular) to the device axis of elongation (A_{D}) may facilitate arresting the advancement of the meniscus and may thus facilitate filling or shaping the (liquid) meniscus. For instance, such a patch may especially provide a transition interface for transition from a (more) attractive surface material to a (more) repelling surface material along the device axis of elongation (A_{D}). Therefore, in embodiments, the liquid meniscus may slow and/or stop at the transition interface. Further, the shape of such a patch may especially affect the shape of the meniscus, such that a more curved patch results in a more curved interface and thus, a higher jet velocity. A plurality of such patches may in embodiments facilitate slowing or stopping the advancement of the liquid meniscus over a region (instead of a single fixed transition interface). In embodiments, such patches may be selected from the group of rectangles (with straight edges), curved shapes (such as a crescent moon or (half) a circle or ellipse), stadium shapes, disk shapes, and triangles (with straight and/or curved edges). Especially, a more curved patch (for example moon shaped, circular shape, etc.) may facilitate ejecting a jet with a smaller jet diameter and a higher jet velocity. Furthermore, a straight-edged patch (for example with straight edges and/or rectangular shaped) may facilitate ejecting a jet with a larger jet diameter and a lower jet velocity. Yet further, a patch with a wave-like structure may (also) facilitate ejecting a jet with a larger jet diameter and a lower jet velocity. Such embodiments are discussed further below.

The patches configured parallel (or nearly parallel) to the device axis of elongation (A_{D}) may affect the (fluid) dynamics of the liquid jet during the jet formation i.e., the shape and/or trajectory of the liquid jet may especially be determined. Such a patch may especially influence the directionality, stability, and repeatability of the liquid jet. In such embodiments, the patch may facilitate confining the jet within the regions with more affinity, especially wherein the directionality of the jet may be defined by said confinement. More especially, the reproducibility of the jet may be improved as a result of suppressing the swaying tail of the (ejected) liquid jet. In embodiments, such patches may be philic and further, such patches may (at least partly) be surrounded by phobic material and may, thus, confine the liquid within regions with more affinity. That is, such a configuration of patches may in embodiments define regions of higher affinity where the motion (or flow) of the liquid may be accelerated (relatively to regions with lower affinity). Furthermore, in embodiments, such (elongated) patches may be configured at an angle with the device angle of elongation (A_{D}), thus facilitating ejecting the liquid jet at an angle to the device angle of elongation. In further embodiments, a plurality of such patches may be configured on the chamber wall to determine the trajectory of the (ejected) liquid jet.

Furthermore, a combination of patches may provide the chamber wall with a heterogenous surface chemistry, and thus, may facilitate providing a stable and reproducible jet. Embodiments comprising such configurations may have:
converging patches (comprising phobic material) with philic regions between said patches, which may provide a jet which may be progressively confined along the device axis of elongation (A_{D}). Such a configuration may provide the advantage of creating a smooth transition towards a small confinement, especially wherein the patches may be curved. Further, in embodiments, patches of triangular or trapezoidal shapes may (also) provide a smooth transition towards a small confinement;
diverging patches (comprising phobic material) with philic regions between said patches may provide a more diffuse jet and a (random) swaying tail. This could be used in applications where a scattered deposition of liquid may be desired;
wave-like patches defined along the device axis of elongation (A_{D}) may increase the number of transitions (between the phobic and philic regions), and thus the jet may be slowed and confined to specific regions.

These aspects are discussed in detail further below.

As mentioned above, in embodiments, the microfluidic device for jet ejection may comprise a hosting chamber defined by a chamber wall. In embodiments, the first chamber end may comprise a first chamber opening. Especially, the first chamber end may comprise a first chamber opening for jet ejection from the hosting chamber. In some embodiments, a part of the first chamber end may comprise the first chamber opening. For instance, in embodiments, the first chamber end may be a closed surface and the first chamber opening may be a hole with dimensions smaller than the first chamber end, and the liquid jet may especially escape via the first chamber opening. However, in other embodiments, the entire first chamber end may comprise the first chamber opening i.e., in embodiments, the cross-sectional area (and shape) of the first chamber opening may be the same as the cross-sectional area (and shape) of the first chamber end.

In embodiments, the hosting chamber may have an elongated shape (or geometry), i.e., one of the dimensions of the hosting chamber (especially the length of the hosting chamber) may be (significantly) larger than the width and the height of the hosting chamber. In particular, in embodiments, the hosting chamber may have a device axis of elongation (A_{D}), especially along the length of the hosting chamber. Especially, along the device axis of elongation (A_{D}), the hosting chamber may have a chamber length (L_{C}). More especially, the chamber length (L_{C}) may be defined between the first chamber end and the second chamber end. The hosting chamber may have a plurality of different shapes and geometries and embodiments of such are described herein.

Especially, the hosting chamber may have a prism or prism-like geometry, especially wherein the hosting chamber may have a shape selected from a square prism, a pentagonal prism, a hexagonal prism, etc. In embodiments, the hosting chamber may have a shape selected from a p-gonal prism, wherein the cross-section of the chamber wall in a plane perpendicular to the device axis of elongation (A_{D}) is a p-sided polygon. In such embodiments, p may be at least 3, such as at least 4, especially at least 5. Further, in embodiments, p may be at maximum 50, such as at maximum 25, especially at maximum 10. In embodiments, the hosting chamber may (also) comprise a circular cross-section (see further below), wherein a circular cross-section may be considered a p-sided polygon where p is infinitely large.

In embodiments, the hosting chamber, such as especially the chamber wall, (in a plane perpendicular to the device axis of elongation (A_{D})) may have a cross-sectional shape comprising equal sides. However, in alternative embodiments, the hosting chamber (in a plane perpendicular to the device axis of elongation (A_{D})) may have a cross-sectional shape comprising unequal sides, for example a rectangular cross-section with a different width and height. In further embodiments, in a cross-section perpendicular to the device axis of elongation (A_{D}), a part of the chamber wall may be curved, for example a rectangular cross-section with two curved sides. Such a geometry may (also) be referred to as rounded rectangular geometry. Furthermore, in embodiments, the hosting chamber may have a circular cylindrical shape, wherein in a plane perpendicular to the axis of the cylinder, the hosting chamber may have a circular cross-section. Further, in embodiments, the hosting chamber may have a cross-sectional shape approximating a shape selected from the group comprising a stadium and an oval.

In embodiments, the hosting chamber may have a chamber height (H_{C}) selected from the range of 1-1000 μm, such as from the range of 2-500 μm, such as from the range of 5-400 μm, especially from the range of 10-200 μm. In embodiments, the hosting chamber may have a chamber width (W_{C}) selected from the range of 1*H_{C}-20*H_{C}, such as from the range of 2*H_{C}-10*H_{C}, especially from the range of 4*H_{C}-8*H_{C}, more especially from the range of 5*H_{C}-7*H_{C}. In embodiments, the hosting chamber may have a chamber length (L_{C}) selected from the range of 10-10000 μm, such as from the range of 100-5000 μm, especially from the range of 500-1000 μm.

Hence, in specific embodiments, the hosting chamber has a chamber height (H_{C}) selected from the range of 5-400 μm, a chamber width (W_{C}) selected from the range of 2*H_{C}-10*H_{C}, and a chamber length (L_{C}) selected from the range of 100-5000 μm, wherein along at least 80% of the chamber length (L_{C}) the hosting chamber has a cross-sectional shape approximating a shape selected from the group comprising a rounded rectangle, a stadium, and an oval.

As mentioned above, the hosting chamber may be configured to host a liquid. In embodiments, the liquid may be water. However, in embodiments, the liquid may (also) be different from water, such as a liquid selected from the group comprising glycerol, alcohols, organic solvents, and (other) hydrocarbons. For instance, in embodiments, the liquid may comprise an oil. Furthermore, in embodiments, the liquid may comprise a dissolved agent, such as a vaccine, or such as insulin. Especially, the liquid may comprise an additive such as a thickener, an emulsifier, a surfactant, a viscoelastic additive, a pigment, and a biomolecule. Furthermore, in embodiments, the liquid may comprise a saline solution. In further embodiments, the liquid may comprise a polypeptide, such as a protein, or such as a cyclic peptide (e.g., cyclosporin). Additionally, in embodiments, the liquid may comprise a skin penetration enhancer, such as an enhancer selected from the group comprising alcohols (for example ethanol), sulfoxides, surfactants (for example sodium dodecyl sulfate), limonene, propanediol, and azone.

In embodiments, the liquid hosted by the hosting chamber may fill part of the hosting chamber and the remainder may be gas (for example air). The liquid may especially have a liquid meniscus i.e., the surface of the liquid in contact with the gas and the chamber wall may be curved. The curvature of the meniscus may be a result of the competing influence of adhesion energy and cohesion energy between the molecules comprised by the liquid and the chamber wall. Especially, the heterogeneous surface chemistry (for example a difference in philicity of different parts (or regions) of the chamber wall) may facilitate the ejection of the liquid from the hosting chamber.

The philicity of a surface may be defined as the ability of a liquid to spread over a surface, wherein the philicity is defined quantitatively by a (equilibrium) contact angle θ (for the liquid). The (equilibrium) contact angle θ may be defined as the angle at which the liquid-gas interface meets the solid-liquid interface i.e., the angle the liquid meniscus makes with the chamber wall. The contact angles specified herein may especially refer to the contact angles of the liquid and the (first/second) surface material at room temperature (20° C.), 1 atm pressure, and where the gas comprises air. An equilibrium contact angle θ<90° indicates that the surface is philic for the liquid, wherein the liquid may spread over a larger area of the surface. An equilibrium contact angle θ>90° indicates that the surface is phobic for the liquid, wherein the liquid may minimize the area of contact with the surface. In embodiments, the phobic surface may have an equilibrium contact angle θ≥91°, such as θ≥93°, especially θ≥95° (for the liquid). In embodiments, the phobic surface may (further) have an equilibrium contact angle θ>91°, such as θ>93°, especially θ>95° (for the liquid). Additionally or alternatively, in embodiments, the philic surface may have an equilibrium contact angle θ≤89°, such as θ≤87°, especially θ≤85° (for the liquid). Further, in embodiments, the philic surface may have an equilibrium contact angle θ<89°, such as θ<87°, especially θ<85° (for the liquid). It will be clear to the person skilled in the art that the equilibrium contact angle θ is dependent on the combination of the surface material and the liquid. Especially, the equilibrium contact angle of a material may vary dependent on the liquid, and especially (also) the contact line (meniscus) behavior of the liquid may be different. Note (also) that the presence of impurities in the liquid may affect the equilibrium contact angle θ.

In embodiments, the chamber wall may comprise a (surface) pattern of a first surface material and a second surface material, wherein the first surface material may have an equilibrium contact angle θ₁ (for the liquid) and the second surface material may have an equilibrium contact angle θ₂ (for the liquid). Especially, the first surface material may be phobic (for the liquid), wherein θ₁>90°. In some embodiments, the second surface material may be philic (for the liquid). However, in other embodiments, the second surface material may also be phobic (for the liquid). In embodiments, the first surface material may (always) have a larger equilibrium contact angle (for the liquid) than the second surface material. Especially, θ₁−θ₂≥20°, such as θ₁−θ₂≥30°, especially θ₁−θ₂≥40°.

In embodiments, at least 1% of the chamber wall, especially of the pattern, comprises the first surface material, such as at least 2%, especially at least 5%. In further embodiments, at least 10% of the chamber wall, especially of the pattern, comprises the first surface material, such as at least 20%, especially at least 30%. In further embodiments, at least 50% of the chamber wall, especially of the pattern, comprises the first surface material, such as at least 60%, especially at least 70%. The term “first surface material” may also refer to a plurality of (different) first surface materials, such as different phobic materials (also see below). For instance, in embodiments wherein a plurality of patches comprise the first surface material, different patches may comprise different first surface materials.

In further embodiments, at least 1% of the chamber wall, especially of the pattern, comprises the second surface material, such as at least 2%, especially at least 5%. In further embodiments, at least 10% of the chamber wall, especially of the pattern, comprises the second surface material, such as at least 20%, especially at least 30%. In further embodiments, at least 50% of the chamber wall, especially of the pattern, comprises the second surface material, such as at least 60%, especially at least 70%. The term “second surface material” may also refer to a plurality of (different) second surface materials, such as different philic materials (also see below). For instance, in embodiments wherein a plurality of patches comprise the second surface material, different patches may comprise different second surface materials.

In further embodiments, the pattern may cover at least 90% of (a surface area of) the chamber wall, such as at least 95%, including 100%. Hence, in embodiments, the first surface material and the second surface material may (together) cover at least 90% of (a surface area of) the chamber wall, such as at least 95%, including 100%.

As mentioned above, the philicity (or “affinity) of the first (or second) surface material may be a function of the liquid hosted by the hosting chamber. In embodiments, the affinity of the liquid to the first (or second) surface material may be a function of the adhesive and cohesive forces between the molecules of the liquid and the first (or second) surface material.

For instance, in embodiments, the liquid may comprise water. In such embodiments, the first surface material and/or the second surface material may comprise a hydrophobic material, especially at least the first surface material may comprise a hydrophobic material. In further embodiments, the hydrophobic material may be selected from the group of polydimethylsiloxane (PDMS), polyvinyl chloride (PVC) and Polytetrafluoroethylene (PTFE). In further embodiments, the hydrophobic material may comprise a hydrophobic coating. For instance, the hydrophobic material may comprise thiol-coated gold. Furthermore, in embodiments, the hydrophobic material may also comprise graphene oxide, especially a graphene oxide coating. Especially, the hydrophobic material may comprise a hydrophobic film, wherein the hydrophobic film may be obtained by depositing graphene oxide (by means of chemical vapor deposition) on the surface of the film. Such films may especially be referred to as highly transparent, flexible and superhydrophobic films (HTFS).

In further embodiments, the second surface material may comprise a hydrophilic material. Especially, the hydrophilic material may be a metal oxide, such as a metal oxide selected from the group comprising Al₂O₃, TiO₂, and SiO₂. In further embodiments, the hydrophilic material may comprise one or more of gold, platinum, chromium, and titanium.

In further embodiments, the first surface material and/or the second surface material may comprise a lipophobic material, especially at least the first surface material may comprise a lipophobic material, or especially at least the second surface material may comprise a lipophobic material. Especially, the lipophobic material may be selected from the group of a fluorosurfactant, a cycloolefin copolymer (COC) and a cyclic olefin polymer (COP). Furthermore, in embodiments, the lipophobic material may comprise a chitosan coating.

In further embodiments, the second surface material may comprise a lipophilic material. Especially, the lipophilic material may be selected from the group comprising a parylene, an alkylsilane (for example octadecylsilane), a polyethylene (for example PTFE) and a polypropylene. Furthermore, in embodiments, the lipophilic material may comprise a thiol coating (for example a dodecane-thiol) or a carbon-based coating.

In embodiments, the (surface) pattern may comprise a (surface) patch. In embodiments, the surface patch may be a two-dimensional shape defined on the (inner) surface of the chamber wall. In embodiments, the patch may comprise one of the first surface material and the second surface material.

In embodiments, the patch may be a two-dimensional region defined by an outline. Especially, the outline may be a single smooth contour, for example an oval. Alternatively, in embodiments, the patch may have an outline defined by a plurality of line segments, for example a rectangle. In further embodiments, at most two of the line segments may be curved, for example a half-stadium. Hence, in embodiments, the patch may have a patch shape approximating a shape (or geometry) selected from the group comprising a triangle, a trapezoid, a rectangle, a crescent moon, an oval, a circle, a wave-like shape, a stadium, and a half-stadium.

In embodiments, the patch may especially have a patch boundary. In embodiments, the patch may be defined (anywhere) on the surface of the chamber wall. Especially, the patch may be defined adjacent to the first chamber end, especially adjacent to the first chamber opening. Hence, in embodiments, at least 50% of the patch boundary may contact the other of the first surface material and the second surface material. In particular, in such embodiments, the remainder of the patch boundary may especially be arranged at the first chamber end, i.e., in embodiments, the patch boundary may define at least part of the first chamber end. In further embodiments, at least 60% of the patch boundary may contact the other of the first surface material and the second surface material. In yet further embodiments, at least 70% of the patch boundary may contact the other of the first surface material and the second surface material. Further, in embodiments, the patch may comprise the first surface material and at least 80% of the patch boundary may contact the second surface material. Analogously, in embodiments, the patch may comprise the second surface material and at least 80% of the patch boundary may contact the first surface material. In yet further embodiments, the patch may contact the other of the first surface material and the second surface material along ≥99% of the patch boundary, including along 100% of the patch boundary.

In specific embodiments, the patch boundary may comprise a plurality of boundary parts. For instance, a (ring-shaped) patch configured on the chamber wall may have a patch boundary comprising two boundary parts. Especially, at least 50% of the patch boundary comprising one or more boundary parts may contact the other of the first surface material and the second surface material.

Alternatively, in embodiments, the patch may be defined away from the first chamber end and the second chamber end. In such embodiments, the patch may comprise one of the first surface material and the second surface material and the entire patch boundary may contact the other of the first material and the second surface material. For instance, in embodiments, the patch comprises the first surface material and the (entire) patch boundary may contact the second surface material. In further embodiments, the patch comprises the second surface material and the (entire) patch boundary may contact the first surface material.

In embodiments, the chamber wall may have a (wall) surface area (S_{W}). In embodiments, the patch may have a (patch) surface area (S_{P}). Especially, the ratio of the surface area of the patch (S_{P}) to the surface area (S_{W}) of the chamber wall may be defined such that 10⁻⁵≤S_{P}/S_{W}≤2*10⁻¹, such as 10⁻⁴≤S_{P}/S_{W}≤2*10⁻¹, especially 10⁻³≤S_{P}/S_{W}≤2*10⁻¹. In embodiments, 10⁻²≤S_{P}/S_{W}≤2*10⁻¹. Further, in embodiments, 10⁻⁵≤S_{P}/S_{W}≤10⁻¹, such as 10⁻⁵≤S_{P}/S_{W}≤10⁻², such as 10⁻⁵≤S_{P}/S_{W}≤10⁻³, especially 10⁻⁵≤S_{P}/S_{W}≤10⁻⁴. In further embodiments, the ratio of the surface area of the patch (S_{P}) to the surface area (S_{W}) of the chamber wall may be defined such that S_{P}/S_{W} is at least 10⁻⁵, such as at least 10⁻⁴, especially at least 10⁻³. In yet further embodiments, the ratio of the surface area of the patch (S_{P}) to the surface area (S_{W}) of the chamber wall may be defined such that S_{P}/S_{W} is at maximum 2*10⁻¹, such as at maximum 2*10⁻², especially at maximum 2*10⁻³. In embodiments, the patch surface area (S_{P}) may be at least 1 μm², such as at least 10 μm², especially at least 100 μm². In further embodiments, the patch surface area (S_{P}) may be at least 0.01 mm², such as at least 0.1 mm², especially at least 1 mm².

The pattern may especially comprise a plurality of patches, wherein each patch comprises the first surface material, or wherein each patch comprises the second surface material. In such embodiments, each patch (of the plurality of patches) may have an independently selected (patch) surface area (S_{P}), such as an independently selected (patch) surface area (S_{P}) for which applies that 10⁻⁵≤S_{P}/S_{W}≤2*10⁻¹, such as 10⁻⁴≤S_{P}/S_{W}≤2*10⁻¹, especially 10⁻³≤S_{P}/S_{W}≤2*10⁻¹ (also see above).

Furthermore, in embodiments, the pattern may have a (pattern) surface area S_{PP}, especially of the first surface material, or especially of the second surface material. Especially, the ratio of the surface area of the pattern (S_{PP}) to the surface area of the chamber wall (S_{W}) may be defined such that 0.01≤S_{PP}/S_{W}≤0.95, such as 0.1≤S_{PP}/S_{W}≤0.95, especially 0.5≤S_{PP}/S_{W}≤0.95. Further, in embodiments, 0.05≤S_{PP}/S_{W}≤0.9, such as 0.05≤S_{PP}/S_{W}≤0.7, especially 0.05≤S_{PP}/S_{W}≤0.5, more especially 0.05≤S_{PP}/S_{W}≤0.3.

In embodiments, the pattern surface area (S_{PP}) may be at least 0.001 mm², such as at least 0.01 mm², especially at least 0.1 mm², more especially 1 mm². In further embodiments, the pattern surface area (S_{PP}) may (even) be at least 5 mm², such as at least 10 mm².

In particular, the (pattern) surface area S_{PP} may refer to a total surface area of the plurality of patches of one material, such as to a total surface area of a plurality of patches comprising the first surface material, or such as to a total surface area of a plurality of patches comprising the second surface material. Hence, in embodiments, the plurality of patches may have a total (pattern) surface area S_{PP}.

Furthermore, as mentioned before, in embodiments, the chamber wall may not necessarily be flat. In embodiments, one or more surfaces of the chamber wall may especially have a curvature. In such embodiments, the patch may (still) be configured (or defined) along the curved chamber wall. Especially, the patch may in such embodiments (also) be curved, such as with the same curvature as the chamber wall.

As mentioned above, the patch may (essentially) be a two-dimensional shape. Hence, the patch may in embodiments have a patch length L_{P} and a patch width W_{P}. In embodiments, the patch may especially be elongated. Especially, a patch axis of elongation (A_{P}) may be defined along the direction of elongation. In embodiments, the patch may have a patch length (L_{P}) along the patch axis of elongation (A_{P}) and the patch may have a patch width (W_{P}) perpendicular to the patch axis of elongation (A_{P}). In embodiments, the patch may have a patch length (L_{P}) larger than the patch width (W_{P}) such that L_{P}≥1.25*W_{P}, such as L_{P}≥1.5*W_{P}, especially L_{P}≥2*W_{P}, more especially L_{P}≥5*W_{P}, more especially L_{P}≥10*W_{P}. In specific embodiments, the patch has a patch axis of elongation (A_{P}), wherein the patch has a patch length L_{P} along the patch axis of elongation (A_{P}) and a patch width W_{P} perpendicular to the patch axis of elongation (A_{P}) (wherein the patch length (L_{P}) and the patch width (W_{P}) are measured along the chamber wall), wherein L_{P}≥1.5*W_{P}.

An elongated shape may especially be beneficial in controlling the advancement of the liquid meniscus. For instance, an elongated patch comprising the second surface material that is philic and oriented along the device axis of elongation (A_{D}) may especially (locally) accelerate the liquid (towards the first chamber opening). Analogously, in embodiments, an elongated patch comprising the first surface material and oriented in direction perpendicular to the device axis of elongation (A_{D}) may arrest the advancement of the liquid meniscus. Hence, in this way, the propagation of the liquid jet may be controlled by the configuration of the patch (i.e., the shape and orientation of the (surface) patch).

In embodiments, the patch may be a contiguous surface i.e., the entire surface area of the patch may be comprised by first surface material or the second surface material. However, in other embodiments, the patch may not be a contiguous surface, especially, the patch may comprise (philic or phobic) dots. In embodiments, the (philic or phobic) dots comprised by each patch may in total have a surface area (S_{D}). In embodiments, S_{D}/S_{P}≥0.3, such as S_{D}/S_{P}≥0.5, especially S_{D}/S_{P}≥0.7. Especially, 0.8≤S_{D}/S_{P}<1.0, such as 0.85≤S_{D}/S_{P}<1.0, especially 0.9≤S_{D}/S_{P}<1.0, more especially 0.95≤S_{D}/S_{P}<1.0. In embodiments, the dots may be circular dots comprising either the first surface material or the second surface material. However, in other embodiments, the dots may not (necessarily) be circular. Especially, the dots may have a shape selected from the group of an oval, an elipse, a square, a half-stadium, and a parallelogram. Furthermore, in embodiments, the dots comprised by each patch may exhibit a polydispersity in size and shape i.e., the dot may have a unique (and different) size and shape. Hence, in this way, the patch surface may especially be covered by dots that may comprise the first surface material or the second surface material.

In embodiments, the patch width W_{P} may be selected from the range of 0.01*L_{C}-0.2*L_{C}, such as 0.05*L_{C}-0.2*L_{C}, especially 0.1*L_{C}-0.2*L_{C}. Further, in embodiments, the patch width W_{P} may be selected from the range of 0.01*L_{C}-0.1*L_{C}, such as 0.01*L_{C}-0.05*L_{C}. In embodiments, the patch axis of elongation (A_{P}) may make an angle α≥80°, such as α≥85°, with the device axis of elongation (A_{D}). Especially, the patch axis of elongation (A_{P}) may (even) be perpendicular to the device axis of elongation (A_{D}) i.e., α=90°. Further, in such embodiments, the patch may especially comprise the first surface material. Hence, in specific embodiments, the patch comprises the first surface material, wherein the patch axis of elongation (A_{P}) makes an angle α≥80° with the device axis of elongation (A_{D}), and wherein the patch width W_{P} is selected from the range of 0.01*L_{C}-0.2*L_{C}. Such a patch may provide the advantage of first arresting the advancement of the meniscus. For instance, the hosting chamber may especially be filled up to a desired volume by strategically configuring an elongated patch along (and perpendicular to) the device axis of elongation (A_{D}) at a predetermined distance from the second chamber end.

In embodiments, the patch may be configured from the second chamber end at a (shortest) distance selected from the range of 0.3*L_{C}-0.9*L_{C}, such as from the range of 0.4*L_{C}-0.8*L_{C}, especially from the range of 0.5*L_{C}-0.7*L_{C}. Further, the patch may be configured nearly perpendicular to the device axis of elongation (A_{D}) and thus arrest the advancement of the meniscus. The volume of liquid hosted by the hosting chamber may influence the characteristics of the ejected liquid jet. Hence, the (elongated) patch (i) comprising the first surface material, (ii) configured nearly perpendicular to the device axis of elongation (A_{D}), and (iii) configured from the second chamber end at a (shortest) distance selected from the range of 0.3*L_{C}-0.9*L_{C} may facilitate providing microfluidic jets with a larger jet volume (i.e., volume of the liquid jet ejected) and/or increased reproducibility.

The term “nearly parallel” in the context of the patch may refer to the patch axis of elongation (A_{P}) making an angle α with the device axis of elongation (A_{D}), wherein α≤30°, such as α≤20°, especially α≤10°.

The term “nearly perpendicular” in the context of the patch may refer to the patch axis of elongation (A_{P}) making an angle α with the device axis of elongation (A_{D}), wherein α≥60°, such as α≥70°, especially α≥80°.

Note that the measurement of the angle α may be measured in the anti-clockwise (or clockwise) direction from the device axis of elongation (A_{D}) to the patch axis of elongation (A_{P}). Hence, the angle α may especially be the smallest angle between the device axis of elongation (A_{D}) and the patch axis of elongation (A_{P}) i.e., no distinction is made between −α and +α, both may be considered α.

In embodiments, the patch length (L_{P}) may be selected from the range of 0.2*L_{C}-0.95*L_{C}, such as from the range of 0.3*L_{C}-0.85*L_{C}, especially from the range of 0.4*L_{C}-0.75*L_{C}, more especially from the range of 0.5*L_{C}-0.65*L_{C}. In embodiments, the patch axis of elongation (A_{P}) may make an angle α≤30°, such as α≤20°, especially α≤10°, with the device axis of elongation (A_{D}). Especially, the patch axis of elongation (A_{P}) may (even) be parallel to the device axis of elongation (A_{D}) i.e., α=0°. Further, in such embodiments, the patch may especially comprise the second surface material. Hence, in specific embodiments, the patch comprises the second surface material, wherein the patch axis of elongation (A_{P}) makes an angle α≤30° with the device axis of elongation (A_{D}), and wherein the patch length (L_{P}) is selected from the range of 0.2*L_{C}-0.95*L_{C}. Such a patch may accelerate the liquid towards the first chamber opening. Especially, such a patch may guide the liquid meniscus along the direction of the patch axis of elongation (A_{P}). More especially, such a patch may be sufficiently long, such that it extends from the first chamber end to a point close to the meniscus of the liquid. Hence, in this way, the liquid jet may be guided along the chamber wall up to the first chamber opening such that the ejected microfluidic jet may have improved stability and reproducibility.

In embodiments, the configuration of the patch may influence the trajectory of the liquid jet and/or the volume of the hosting chamber up to which the liquid may be filled. Especially, the patch may be defined on the surface of the hosting chamber. Hence, in some instances, a part of the chamber wall may comprise the patch, however, the part of the chamber wall opposite to the said patch may not (necessarily) comprise a second patch. This may result in only a part of the meniscus being arrested (i.e., the liquid meniscus is only arrested on one side of the chamber wall) and/or the trajectory of the liquid jet being skewed (i.e., the liquid jet may be disproportionately accelerated on one side of the chamber wall). This may be mitigated by the symmetric arrangement of one or more patches on the chamber wall. Hence, in embodiments, the pattern may comprise one or more patches, especially wherein the patches may be configured on the chamber wall such that the one or more patches are arranged symmetrically on the chamber wall.

In some embodiments, during the operation of the microfluidic device, the liquid jet may be ejected at an angle from the first chamber opening. This may be controlled (or mitigated) by the configuration of (just) one patch in embodiments, wherein the patch may be configured to (at least partially) determine the trajectory of the ejected liquid jet. Hence, in this way, even a single patch may facilitate improving the stability and the reproducibility of the liquid jet. Furthermore, in embodiments, (even) the pattern comprising one (single) patch may have a plane of symmetry, for example, the plane of symmetry may pass through the device axis of elongation and the centroid of the patch may lie in the plane of symmetry.

Hence, in embodiments, the one or more patches may be configured such that the patches have a plane of symmetry, especially, the plane of symmetry may pass through and be parallel to the device axis of elongation (A_{D}). Additionally or alternatively, in embodiments, the patches may (also) be configured such that the patches have a plane of symmetry, especially, the plane of symmetry may be perpendicular to the device axis of elongation (A_{D}). Hence, in specific embodiments, it applies that the pattern (of the first surface material and the second surface material) has a plane of symmetry (wherein the device axis of elongation (A_{D}) is coincident with the plane of symmetry, or wherein the device axis of elongation (A_{D}) is to the plane of symmetry).

In embodiments, in a plane perpendicular to the device axis of elongation (A_{D}) the chamber wall (comprised by the hosting chamber) may have a perimeter with a (chamber) perimeter length P_{C}. In embodiments wherein the hosting chamber has a cylindrical geometry, the circumference of the chamber wall in a cross-section perpendicular to the device axis of elongation (A_{D}) may have a chamber (boundary) length P_{C}.

As mentioned before, the patch may in embodiments be defined on the chamber wall in any orientation i.e., the patch axis of elongation (A_{P}) may be aligned at an angle α with the device axis of elongation (A_{D}). Therefore, in a cross-section perpendicular to the device axis of elongation (A_{D}) a part of the chamber wall may especially be covered by the patch. Especially, the patch may cover a (patch) perimeter length (P_{P}) along the perimeter of the chamber wall having a (chamber) perimeter length (P_{C}). In embodiments, the patch may be configured perpendicular to the device axis of elongation (A_{D}) and the (patch) perimeter length (P_{P}) may be equal to the patch length (L_{P}) in a cross-section perpendicular to the device axis of elongation (A_{D}). Further, in embodiments, the patch may be configured parallel to the device axis of elongation (A_{D}) and the (patch) perimeter length (P_{P}) may be equal to the patch width (W_{P}) in a cross-section perpendicular to the device axis of elongation (A_{D}). In other embodiments, the patch axis of elongation (A_{P}) may be configured at an angle α to the device axis of elongation (A_{D}) and the (patch) perimeter length (P_{P}) may be in the range of W_{P}-L_{P} in a cross-section perpendicular to the device axis of elongation (A_{D}).

In embodiments, in a cross-section perpendicular to the device axis of elongation (A_{D}), the patch may cover a part of the chamber wall. Especially, the patch axis of elongation (A_{P}) may be configured parallel or nearly parallel to the device axis of elongation (A_{D}). Hence, in embodiments, the (patch) perimeter length (P_{P}) may be selected from the range of 0.01*P_{C}-0.5*P_{C}, such as 0.05*P_{C}-0.25*P_{C}, especially 0.1*P_{C}-0.2*P_{C}. Hence, in specific embodiments, in a cross-section perpendicular to the device axis of elongation (A_{D}) the hosting chamber has a perimeter (or “circumference”) with a (chamber) perimeter length (P_{C}), wherein the patch covers a (patch) perimeter length (P_{P}) along the perimeter, wherein P_{P} is selected from the range of 0.05*P_{C}-0.25*P_{C}.

A patch covering a (patch) perimeter length (P_{P}) selected from the range of 0.01*P_{C}-0.5*P_{C}, such as especially from the range of 0.05*P_{C}-0.25*P_{C}, may provide control over the thickness (and/or volume) of the microfluidic jet. Further, as the patch may cover (only) part of a (chamber) perimeter length P_{C}, the (microfluidic) jet may be accelerated differently on the side of the chamber wall covered by the patch compared to the side of the chamber wall not covered by the patch, facilitating controlling the trajectory of the microfluidic jet, such as e.g. facilitating controlling a direction (e.g. an angle) of ejection of the microfluidic jet from the first chamber opening. Further, a patch covering a (patch) perimeter length (P_{P}) selected from the range of 0.01*P_{C}-0.5*P_{C}, such as especially from the range of 0.05*P_{C}-0.25*P_{C}, may provide control over the shape (and location) of the meniscus. That is, a part of the chamber wall may comprise the patch (having a perimeter length (P_{P}) selected from the range of 0.01*P_{C}-0.5*P_{C}), while another part of the chamber wall (having a length of 0.5*P_{C}-0.99*P_{C} in a cross-section perpendicular to the device axis of elongation (A_{D})) may not (necessarily) comprise a patch. This may result in only a part of the meniscus being arrested (i.e., the liquid meniscus is only arrested on one side of the chamber wall), hence, the meniscus may have an (a)symmetrical shape determined by the (patch) perimeter length (P_{P}) of the patch, which may provide further options for controlling the properties of the microfluidic jet.

Especially, the patch axis of elongation (A_{P}) may be configured perpendicular or nearly perpendicular to the device axis of elongation (A_{D}). In embodiments, the (patch) perimeter length (P_{P}) may be selected from the range of 0.2*P_{C}-P_{C}, such as from the range of 0.4*P_{C}-P_{C}, especially from the range of 0.6*P_{C}-P_{C}. Hence, in specific embodiments, in a cross-section perpendicular to the device axis of elongation (A_{D}) the hosting chamber has a perimeter (or “circumference”) with a (chamber) perimeter length (P_{C}), wherein the patch covers a (patch) perimeter length (P_{P}) along the perimeter, wherein P_{P} is selected from the range of 0.4*P_{C}-P_{C}.

During the application of the microfluidic device, the liquid may have to be controlled, for instance the liquid may have to be filled to a predetermined volume of the hosting chamber. Further, during the application of the microfluidic device, energy may be provided to the liquid causing the microfluidic jet to be ejected from the microfluidic device via the first chamber opening. Hence, with such a configuration of the patch (on the chamber wall), the chamber wall may in embodiments have a heterogenous surface chemistry (i.e., a difference in philicity). Especially, such a heterogenous surface chemistry may facilitate ejecting a stable and reproducible microfluidic jet.

As mentioned before, the pattern may in embodiments comprise a plurality of patches. In embodiments, the pattern may comprise a first set of n patches. In embodiments, each patch of the first set may comprise the first surface material. In other embodiments, each patch of the first set may comprise the second surface material.

In embodiments, n may be at least two, such as at least five, especially at least ten. In embodiments, the n patches may be arranged successively (in a direction parallel to the device axis of elongation (A_{D}) downstream of the second chamber end. Here, two patches configured successively may refer to a second patch configured downstream of a first patch. Likewise, the n patches may in embodiments be configured successively i.e., each patch of the first set may be configured downstream of the preceding patch of the first set (with the exception of the first patch configured closest to the second chamber end). In embodiments, the distance between any two successive patches of the first set may be the same i.e., the patches of the first set may be spaced equidistantly. However, in other embodiments, the distance between any two successive patches of the first set may (also) be different. In embodiments, the patches of the first set may be configured perpendicular or nearly perpendicular to the device axis of elongation (A_{D}). Especially, the patch axis of elongation (A_{P}) of each patch of the first set may make an angle α≥80°, such as α≥85°, especially α=90° with the device axis of elongation (A_{D}).

Note that, the terms “upstream” and “downstream”, such as in the context of ejection of the liquid jet may especially relate to an arrangement of items or features relative to the device axis of elongation (A_{D}), wherein relative to a first position along the device axis of elongation (A_{D}), a second position along the device axis of elongation (A_{D}) closer to the second chamber end (than the first position) is “upstream”, and a third position along the device axis of elongation (A_{D}) further away from the second chamber end (than the first position) is “downstream”.

Hence, in specific embodiments, the pattern comprises a first set of n patches, wherein n≥2, wherein the n patches are arranged successively (and equidistantly) (in a direction parallel to the device axis of elongation (A_{D})) downstream from the second chamber end, wherein each patch of the first set comprises the first surface material, wherein the patch axis of elongation (A_{P}) of each patch of the first set makes an angle α≥80° with the device axis of elongation (A_{D}).

During the application of the microfluidic device, energy may be supplied to the liquid resulting in the ejection of the microfluidic jet. As the liquid meniscus advances along the direction of the device axis of elongation (A_{D}) the one or more patches of the first set may comprise the first surface material and hence, may hinder the advancement of the liquid meniscus. Especially, such an arrangement of the first set of patches may successively reduce the velocity at which the microfluidic jet is ejected. Hence, the velocity of the microfluidic jet may especially be controlled by the number of patches, the orientation angle of the patches, the equilibrium contact angle of each patch and the distance between successive patches.

In embodiments, the pattern may comprise a second set of k patches. In embodiments, each patch of the second set may comprise the second surface material.

In embodiments, k may be at least two, such as at least five, especially at least ten. In embodiments, each patch of the second set may be configured at a (shortest) distance selected from the range of 0.3*L_{C}-0.9*L_{C}, such as from the range of 0.3*L_{C}-0.75*L_{C}, such as from the range of 0.3*L_{C}-0.6*L_{C}, from the second chamber end (measured in a direction along the device axis of elongation (A_{D})). Further, in embodiments, each patch of the second set may be configured at a (shortest) distance selected from the range of 0.4*L_{C}-0.9*L_{C}, such as from the range of 0.5*L_{C}-0.9*L_{C}, such as from the range of 0.6*L_{C}-0.9*L_{C}, from the second chamber end (measured in a direction along the device axis of elongation (A_{D})). In embodiments, the (shortest) distance from the second chamber end may be the same for each of the k patches from the second set. Alternatively, the (shortest) distance from the second chamber end may be individually selected (such as from the range of 0.3*L_{C}-0.9*L_{C}, see also above) for each of the k patches from the second set.

In embodiments, the patches of the second set may be configured parallel or nearly parallel to the device axis of elongation (A_{D}). Especially, the patch axis of elongation (A_{P}) of each patch of the second set may make an angle α≤30°, such as α≤20°, especially α≤10°, more especially α=0°, with the device axis of elongation (A_{D}). Further, in embodiments, the patches of the second set may converge or diverge towards the first chamber end.

Hence, in specific embodiments, the pattern comprises a second set of k patches, wherein k≥2, wherein each patch of the second set comprises the second surface material, wherein each patch of the second set is configured at a (shortest) distance selected from the range of 0.3*L_{C}-0.9*L_{C} from the second chamber end (measured along the device axis of elongation (A_{D})), wherein the patch axis of elongation (A_{P}) of each patch of the second set makes an angle α≤30° with the device axis of elongation (A_{D}).

As mentioned before, the patch may in embodiments be a two-dimensional shape. Hence, the shortest distance may be measured from the point on the outline of the patch closest to the second chamber end to the second chamber end.

During the application of the microfluidic device, energy may be supplied to the liquid resulting in the ejection of the microfluidic jet. The second set of patches may in embodiments comprise the second surface material. Especially, the second surface material may be philic and hence, the second set of patches may be configured to accelerate and guide the liquid jet as it is ejected from the microfluidic device. Hence, as mentioned above, the second set of patches may, in embodiments, be configured closer (shortest distance) to the first chamber end than to the second chamber end. Further, the affinity of the liquid for the second surface material may in embodiments provide the benefit of (relatively) accelerating the liquid along the patch. Yet further, the patches of the second set may (i) be configured parallel or nearly parallel to the device axis of elongation (A_{D}), and (ii) converge or diverge towards the first chamber end, thus directing the jet along a predefined trajectory such that a stable and reproducible jet may be ejected. Hence, the velocity of the microfluidic jet may especially be controlled by the number of patches, the shape(s) of the patches, the orientation angle of the patches, the contact angle of each patch and the distance between successive patches.

In embodiments, the pattern may comprise a third set of m patches. In embodiments, m may be at least two, such as at least five, especially at least ten. In embodiments, each patch of the third set may comprise the second surface material.

In embodiments, the centroid of (all) the patches of the third set may lie along an axis (A_{M}) defined on the chamber wall. Especially, the axis (A_{M}) may be parallel to the device axis of elongation (A_{D}). In embodiments, the patch axis of elongation (A_{P}) of each patch of the third set may each make an angle α with the device angle of elongation (A_{D}) (and the axis (A_{M})). In embodiments, (all) patches of the third set may make the same angle α with the device axis of elongation (A_{D}). However, in other embodiments, each patch of the third set may (also) make a different angle α with the device angle of elongation (A_{D}). In embodiments, the patch axis of elongation (A_{P}) of the m patches of the third set may make an angle 5°≤α≤50°, such as 5°≤α≤40°, especially 5°≤α≤30° with the device axis of elongation (A_{D}). Further, in embodiments, the patch axis of elongation (A_{P}) of the m patches of the third set may make an angle 10°≤α≤30°, such as 15°≤α≤30°, especially 20°≤α≤30°, more especially 25°≤α≤30° with the device axis of elongation (A_{D}).

Further, in embodiments, the third set of patches may be configured successively along the axis (A_{M}). In embodiments, the distance between any two consecutive patches of the third set may be the same i.e., the patches of the third set may be configured equidistant. In other embodiments, the distance between two consecutive patches may be different. Especially, the centroids of two consecutive patches of the third set may be configured at a first distance d1. In embodiments, d1 may be selected from the range of 0.05*L_{C}-0.5*L_{C}, such as from the range of 0.1*L_{C}-0.4*L_{C}, especially from the range of 0.2*L_{C}-0.3*L_{C}. Note that in this context, the phrase “consecutive” is used interchangeably with the term “successive”.

In embodiments, the pattern may comprise a plurality of such third sets. Hence, in specific embodiments, the pattern comprises a plurality of third sets, wherein each third set comprises a set of m patches, wherein m≥2, wherein each patch of the third set comprises the second surface material, wherein the centroid of (all) the patches lie along an axis (A_{M}) defined on the chamber wall, wherein the axis (A_{M}) is parallel to the device axis of elongation (A_{D}), wherein the patch axis of elongation (A_{P}) of the m patches of the third set makes an angle 5°≤α≤50° with the device axis of elongation (A_{D}), wherein the centroids of (each) two consecutive patches of the third set are configured at a first distance d1, wherein d1 is selected from the range of 0.05*L_{C}-0.5*L_{C}.

In embodiments comprising a plurality of third sets, the centroids of all patches of each third set may lie along a unique axis (A_{M}), i.e., each of the third sets may have a respective axis A_{M}. Especially, the axis (A_{M}) may be defined on the chamber wall such that the distance between two consecutive axes (A_{M}) may be a distance d2 (measured in a perpendicular direction to the axis (A_{M}) and along the chamber wall). Especially, d2 may be selected from the range of 0.2*P_{C}-0.5*P_{C}, such as from the range of 0.25*P_{C}-0.45*P_{C}, especially from the range of 0.3*P_{C}-0.4*P_{C}.

Hence, in specific embodiments, the axes (A_{M}) of two neighboring third sets are arranged at a distance d2, wherein d2 (measured in a perpendicular direction to axis (A_{M}) and along the chamber wall) is selected from the range of 0.2*P_{C}-0.5*P_{C}.

Such an arrangement of patches (such as the plurality of the third set of patches) may facilitate guiding and controlling the microfluidic jet towards the first chamber opening. Such control of the microfluidic jet may facilitate providing a stable and reproducible jet.

In a further aspect, the invention may provide a jet ejection system comprising the microfluidic device of the invention, a liquid supply, and a heating system. Especially, the hosting chamber may comprise a hosting chamber opening. The liquid supply may in embodiments be configured to provide the liquid to the hosting chamber via the hosting chamber opening. In embodiments, the heating system may be configured to provide radiation to one or more of the chamber wall and/or the liquid in the hosting chamber. In specific embodiments, the invention provides a jet ejection system comprising (i) the microfluidic device, (ii) a liquid supply and (iii) a (laser-based) heating system, wherein the hosting chamber comprises a hosting chamber opening, wherein the liquid supply is configured to provide the liquid to the hosting chamber via the hosting chamber opening, wherein the (laser-based) heating system is configured to provide (laser) radiation to one or more of the chamber wall and the liquid in the hosting chamber, especially wherein the laser radiation comprises an infrared laser pulse.

The jet ejection system may especially facilitate the ejection of the microfluidic jet via the first chamber opening. In embodiments, some of the functions carried out by the jet ejection system may be first, filling the hosting chamber with the liquid to a predetermined volume, and second, providing energy to eject the microfluidic jet via the first chamber opening.

The microfluidic device may in embodiments comprise the pattern comprising one or more patches, especially a plurality of patches each comprising the first surface material. Such a configuration may facilitate filling the hosting chamber with a predefined volume of the liquid. In particular, such configuration may facilitate providing a meniscus at a predefined location (along the device axis of elongation (A_{D})).

Further, in embodiments, the hosting chamber may comprise the hosting chamber opening. Especially, the hosting chamber opening may be located at a distance L_{O} from the second chamber end. In embodiments, L_{O} may be selected from the range of 10-2000 μm, such as from the range of 100-1500 μm, especially from the range of 500-1000 μm. Further, in embodiments, the hosting chamber opening may have a diameter D_{O}. Especially, D_{O} may be selected from the range of 1-1000 μm, such as from the range of 10-500 μm, especially from the range of 25-300 μm, such as from the range of 25-200 μm. In embodiments, L_{O} may be defined such that 0≤L_{O}/L_{C}≤0.6, such as 0.1≤L_{O}/L_{C}≤0.5, especially 0.2≤L_{O}/L_{C}≤0.4. Note that in some embodiments, the hosting chamber opening may (also) be configured in the second chamber end, i.e., L_{O}=0.

In embodiments, the hosting chamber may be fluidically coupled to the liquid supply i.e., the liquid may be flowed to the hosting chamber from the liquid supply. Hence, in embodiments, tubes or (micro) pipes may be used to connect the liquid supply to the hosting chamber opening. In this way, the liquid may be flowed into the hosting chamber via the hosting chamber opening.

In embodiments, the jet ejection system may provide energy to the microfluidic device, especially to the liquid hosted in the hosting chamber. Energy may in embodiments be supplied in a plurality of different ways to the liquid. In embodiments, the hosting chamber (especially, the region close to the second chamber end) may be heated by means of a heating system, which may comprise a laser, a gas burner, electric resistance heating, etc. Furthermore, in embodiments, the chamber wall may (also) be heated by means of a thermal heater or a (high-temperature) heating element.

Furthermore, in embodiments, heat may be provided by means of optical (or laser) radiation. That is, by directing a laser beam on to the chamber wall, especially in a region close to the second chamber end. Especially, the laser radiation may comprise an infrared laser pulse. Alternatively, in embodiments, the laser radiation may be provided to one end of an optic fiber, such that the laser radiation is transmitted (by means of total internal reflection) to the other end of the optic fiber, wherein the other end of the optic fiber is attached to or configured close to the chamber wall (especially the second chamber end). In embodiments, the chamber wall may be transmissive for optical radiation. Hence, in this way, energy may be provided to the liquid by means of optical (or laser) radiation. Especially, the wavelength of the (laser) radiation provided may be selected from the range of 200-11000 nm, such as from the range of 300-8000 nm, especially from the range of 400-3000 nm. Additionally or alternatively, the chamber wall may in embodiments comprise a thermal coating (applied on the outside of the chamber wall). Especially, the thermal coating may be heated by means of optical (or laser) radiation and the heat may be conducted via the chamber wall to the liquid. Typically, in embodiments, the energy provided may be in the range of 0.01-50 mJ.

Alternatively, in embodiments, mechanical energy may be provided to the liquid. For instance, in some embodiments, the second chamber end may comprise a second chamber opening. Especially, a piston may be configured within the hosting chamber (and extending into the hosting chamber via the second chamber opening) such that the piston may be actuated along the device axis of elongation (A_{D}). Hence, in this way, the piston may be advanced to advance the liquid in the hosting chamber. Especially, the piston may have dimensions equal to the (inner) dimensions of the hosting chamber, such that the liquid may not flow upstream of the piston as the piston is advanced. The piston may in embodiments have a height (H_{T}), wherein H_{T} may be selected from the range of 1-1000 μm, especially from the range of 2-500 μm, such as from the range of 5-400 μm, especially from the range of 10-200 μm. Further, the piston may in embodiments have a width (W_{T}), wherein W_{T} may be selected from the range of 1*H_{T}-20*H_{T}, such as from the range of 2*H_{T}-10*H_{T}, especially from the range of 4*H_{T}-8*H_{T}, more especially from the range of 5*H_{T}-7*H_{T}. In embodiments, the piston may be actuated by means of a compressed spring configured outside the hosting chamber (or alternatively in certain embodiments within the second chamber end). The spring may especially be compressed and when released may drive the piston in a downstream direction. Further, in embodiments, the piston may be actuated by means of (pressurized) gas. Especially, pressurized gas may be supplied upstream of the piston and the expansion of the gas may actuate the piston in a downstream direction.

In embodiments, a piezo electric actuator may be used for jet ejection. In particular, the microfluidic device may comprise a piezo electric actuator configured to provide (mechanical) energy to the liquid. Especially, in embodiments, the piston (see above) may comprise a piezo electric actuator.

Alternatively, in embodiments, pressurized gas may be provided to the hosting chamber (in the absence of a piston). Especially, the chamber wall may comprise a second hosting chamber opening. Especially, the second hosting chamber opening may be located at a distance L_{O2} from the second chamber end. In embodiments L_{O2} may be selected from the range of 10-2000 μm, such as from the range of 100-1500 μm, especially from the range of 500-1000 μm. Further, in embodiments, the second hosting chamber opening may have a diameter D_{O2}. Especially, D_{O2} may be selected from the range of 1-500 μm, such as from the range of 5-250 μm, especially from the range of 10-100 μm. In embodiments, the hosting chamber may be fluidically coupled to a gas supply, i.e., in embodiments, the gas supply may be configured to provide a (pressurized) gas to the hosting chamber via the second hosting chamber opening. Hence, in embodiments, tubes or (micro) pipes may be used to connect the gas supply to the second hosting chamber opening. Pressurized gas may be flowed into the hosting chamber, especially at a location close to the second chamber end. Especially, the pressurized gas may expand and thus force the liquid along the device axis of elongation (A_{D}) in a downstream direction, thus ejecting the microfluidic jet.

Hence, in this way, energy may be supplied to the liquid by a plurality of different ways (for example: thermal, mechanical, optical, etc.).

In further embodiments, the jet ejection system may comprise a control system. Especially, the control system may be configured to control (or operate) the jet ejection system. Especially, the control system may control the filling of the liquid in the hosting chamber. That is, the control system may in embodiments regulate the flow of liquid from the liquid supply to the hosting chamber. Further, the control system may regulate the energy provided to the liquid in the hosting chamber. For instance, the control system may regulate the gas pressure provided to an embodiment of the microfluidic device. Alternatively, in embodiments, the control system may regulate the power of the laser-based radiation system. Hence, in this way, the control system may especially control the operation of the jet ejection system.

The term “controlling” and similar terms herein may especially refer at least to supervising the operation of the jet ejection system. Hence, herein “controlling” and similar terms may refer to supervising the operation of the jet ejection system or one or more elements comprised by the jet ejection system. The controlling of said element can be done with a control system. The control system and the element may thus at least temporarily, or permanently, be functionally coupled. The element may comprise the control system. In embodiments, the control system and the element may not (necessarily) be physically coupled.

Control can be done via wired and/or wireless control. The term “control system” may also refer to a plurality of different control systems, which especially are functionally coupled, and of which e.g. one master control system may be a control system and one or more others may be slave control systems. In embodiments, the control system may be operated via a user interface, though other options, like executing an operation in dependence of an (external) sensor signal or a (time) scheme, may also be possible.

In a further aspect, the invention may provide a method for ejecting a jet using the microfluidic device. In embodiments, the method may comprise a liquid provision step comprising providing the liquid to the hosting chamber. Especially, the liquid provision step may comprise filling 20-70 vol. % of the hosting chamber with the liquid. In embodiments, the method may (also) comprise an ejection step comprising providing radiation to the chamber wall and/or to the liquid such that at least part of the liquid is boiled and a liquid jet is ejected.

Hence, in specific embodiments, the invention may provide a method for ejecting a jet with the microfluidic device, wherein the method comprises a liquid provision step comprising providing the liquid to the hosting chamber, wherein the liquid provision step comprises filling 20-70 vol. % of the hosting chamber with the liquid, wherein the method comprises an ejection step comprising providing radiation to the chamber wall and/or to the liquid such that at least part of the liquid is boiled and a liquid jet is ejected.

As mentioned above, the level to which the liquid is filled in the hosting chamber may influence the stability and the reproducibility of the microfluidic jet ejected from the system. Especially, the volume of the hosting chamber filled with the liquid may be dependent on the configuration of the pattern comprised by the chamber wall. That is, depending on the configuration of the one or more patches (or sets of patches) comprised by the pattern, the volume percentage of the hosting chamber filled with the liquid may be different. In embodiments, the liquid provision step may comprise filling 10-80 vol. %, such as 20-70 vol. %, especially 30-60 vol. %, more especially 40-50 vol. %, of the hosting chamber (volume) with the liquid.

Further, in embodiments, the method may comprise an ejection step. In embodiments, the ejection step may comprise providing radiation to the chamber wall and/or to the liquid. In embodiments, the radiation may be provided by means of a laser-based source.

As mentioned above, in embodiments, the chamber wall may be transmissive for optical radiation. Hence, energy may be provided to the liquid by means of optical (or laser) radiation. Especially, the liquid may absorb the (optical) energy and may be heated. More especially, a part of the liquid may be boiled (or vaporized) to form a bubble, wherein the expanding bubble may facilitate the ejection of the liquid. Additionally or alternatively, the laser may be focused onto a part of the chamber wall and the heat may be conducted via the chamber wall to the liquid. Especially, the chamber wall may comprise the thermal coating, wherein the thermal coating may facilitate the conduction of heat to the liquid. Hence, the method may especially comprise focusing the laser on a part of the chamber wall closer to the second chamber end than the first chamber end. Furthermore, in embodiments, an optic fiber may be used, wherein the laser may be directed to be incident on one end of the optic fiber and transmitted by means of total internal reflection to the other end of optic fiber, which may be in contact with or configured in the vicinity of the chamber wall. Hence, in this way, laser-based radiation may be provided to the liquid. In embodiments, radiation may be provided to the chamber wall and/or to the liquid such that at least part of the liquid is boiled (and/or vaporized) and a liquid jet is ejected.

Alternatively, in embodiments, the method may comprise an ejection step, wherein mechanical energy may be provided to the liquid. Especially, the method may comprise actuating a piston configured within the hosting chamber. In embodiments, the method may comprise actuating the piston by means of a compressed spring configured outside the hosting chamber (or alternatively in certain embodiments within the hosting chamber). The method may especially comprise releasing the (compressed) spring, thus driving the piston in a downstream direction. Further, in embodiments, the method may comprise actuating the piston by providing (pressurized) gas upstream of the piston. Especially, the expansion of the gas may actuate the piston in a downstream direction.

Alternatively, in embodiments, the method, especially the ejection step, may comprise providing pressurized gas to the hosting chamber (even in the absence of a piston). Especially, (pressurized) gas may be flowed to the hosting chamber from the gas supply, and thus force the liquid along the device axis of elongation (Ap), thus ejecting the microfluidic jet.

As mentioned before, the ejection step may comprise boiling and/or vaporizing part of the liquid (in the hosting chamber) by heating the liquid. Especially, heat may be provided at a power selected from the range of 0.10-10 W, such as from the range of 0.15-8 W, especially from the range of 0.20-5 W, more especially from the range of 0.25-1.5 W.

Jet ejection systems and methods are known in the art. For instance, the method of the invention may be performed by ejecting a liquid jet on a target material with the system described in WO2020182665, which is hereby herein incorporated by reference.

Note that the target material may especially be a viscoelastic material, i.e., a material that exhibits both elastic and viscous behavior when deformed. For instance, in embodiments, the target material may comprise a polymer. In further embodiments, the target material may comprise (ex vivo) soft tissue, such as (ex vivo) skin, or such as an (ex vivo) eye. In further embodiments, the target material may comprise a hydrogel, such as one or more of gelatin, agarose and a polyacrylamide. Gelatin and agarose may be commonly used to give texture to foods and also as skin surrogates. Polyacrylamide may, for instance, be used for studies on cell durotaxis (the ability of cells to move in a substrate with a stiffness gradient). In further embodiments, the target material may comprise an artificially created tissue or a biomaterial, such as a dermal equivalent or a cell cultivation tissue for an implant, for example 3D printed tissues and organs.

Hence, in embodiments, the target material may be inanimate. Further, in embodiments, the method may be a non-medical method.

In further embodiments, the target material may comprise a tissue, such as skin tissue or eye tissue, especially of a subject.

In embodiments, the method may comprise ejecting liquid, especially a liquid jet, with a jet velocity selected from the range of 1-250 m/s, such as from the range of 2-150 m/s, especially from the range of 5-70 m/s. The liquid jet may especially be incident on a target comprising the target material. It will be clear to the person skilled in the art that the suitable jet velocities may depend on jet properties of the liquid jet, such as jet diameter, jet volume and/or jet angle (to the target material). Generally, the jet velocity of the liquid jet may be (relatively) stable from the moment of ejection to the moment of impact on the target material. The jet velocities mentioned herein may, however, especially refer to the jet velocity of the liquid jet right before impact of the liquid jet on the target material.

In further embodiments, the method may comprise ejecting the liquid jet with a jet volume selected from the range of ≤500 μl, such as from the range of ≤200 μl, especially from the range of ≤100 μl. In further embodiments, the liquid jet may be ejected with a jet volume selected from the range of ≤10 μl, such as from the range of ≤5 μl, especially from the range of ≤1 μl. In further embodiments, the liquid jet may be ejected with a jet volume selected from the range of 2-50 nl, such as from the range of 5-25 nl, especially from the range of 8-13 nl. In particular, a (relatively) low jet volume may be selected to avoid a pooling of liquid on the target material. Hence, in further embodiments, the liquid jet may be provided with a jet volume selected from the range of ≤75 nl, such as from the range of ≤50 nl, especially from the range of ≤40 nl. In further embodiments, the liquid jet may be provided with a jet volume selected from the range of ≤30 nl, such as from the range of ≤20 nl, especially from the range of ≤15 nl.

In further embodiments, the method may comprise ejecting the liquid jet with a circular equivalent (jet) diameter selected from the range of 20 μm-5 mm, such as from the range of 30 μm-3 mm, especially from the range of 50 μm-1 mm. In embodiments, the circular equivalent diameter may especially be ≤3 mm, such as ≤1 mm, especially ≤500 μm, such as ≤100 μm. The equivalent circular diameter (or ECD) (or “circular equivalent diameter”) of an (irregularly shaped) two-dimensional shape is the diameter of a circle of equivalent area. For instance, the equivalent circular diameter of a square with side a is 2*a*SQRT(1/π). For a circle, the diameter is the same as the equivalent circular diameter. Would a circle in an xy-plane with a diameter D be distorted to any other shape (in the xy-plane), without changing the area size, then the equivalent circular diameter of that shape would be D.

In general, the liquid jet may be provided perpendicularly to the target material, i.e., the liquid jet may travel along a path (essentially) perpendicular to the target material before impacting the target material. In further embodiments, the jet may be provided at a (jet) angle, wherein the jet angle is the angle that the jet makes with the surface of the target material at the point of impact of the jet on the target material. In embodiments, the liquid jet may be ejected towards the target material at an (jet) angle of 45°-90° (to the target material), such as at an angle of 60-90°, especially at an angle of 75-90°, such as at an angle of 85-90°, especially (essentially) 90°. In embodiments, the method may comprise providing the liquid jet to the target material at an angle of 30°-85° (to the target material), such as at an angle of 45-80°, especially at an angle of 50-75°.

Further, in embodiments, the method may comprise varying a jet property of the liquid jet (over time). For instance, the method may comprise varying the (circularly or spherically equivalent) diameter or jet velocity of the liquid jet over time. In particular, in embodiments, the method may comprise varying one or more of the jet velocity, the (circular or spherical equivalent) diameter, and the jet angle (relative to the target material) of the liquid jet.

As described above, a plurality of successively provided droplets may be considered a liquid jet. In particular, in embodiments, the method may comprise providing the liquid jet, wherein the liquid jet may comprise one or more (successively provided) droplets. In further embodiments, the (liquid) droplet may be ejected with a spherical equivalent diameter selected from the range of 30 μm-3 mm, such as from the range of 50 μm-2 mm, especially from the range of 100 μm-1 mm. The equivalent spherical diameter (or ESD) (or “spherical equivalent diameter”) of an (irregularly shaped) three-dimensional shape is the diameter of a sphere of equivalent volume. For a sphere, the diameter is the same as the equivalent spherical diameter. Would a sphere in an xyz-plane with a diameter D be distorted to any other shape (in the xyz-plane), without changing the volume, then the equivalent spherical diameter of that shape would be D.

Further, in embodiments, (droplets in) the plurality of droplets may be provided at intervals (independently) selected from the range of 0.02-10 ms, such as selected from the range of 0.05-5 ms, especially from the range of 0.1-2 ms. Hence, in embodiments, the method may comprise providing the plurality of droplets at a frequency selected from the range of 0.001-50 kHz, such as from the range of 0.1-50 kHz, especially from the range of 1-30 KHz.

In particular, in embodiments, the method may comprise varying the properties of two or more of the plurality of droplets, such as by continuously, especially linearly, changing a property of the droplets, or such as by step-wise changing a property of the droplets (e.g., droplets with (essentially) a first set of properties followed by droplets with a second set of properties).

Hence, in embodiments, the method may comprise varying a jet property of the liquid jet (over time) by varying the jet property along the plurality of (successively provided) droplets. In further embodiments, the method may comprise varying the intervals between two or more successive droplets of the plurality of droplets, i.e., two or more successive intervals may differ in duration. Especially, the method may comprise varying the frequency at which the plurality of droplets are provided (over time). In particular, in embodiments, the method may comprise varying the frequency in the range of 0.1-50 kHz. In further embodiments, the method may comprise providing a frequency sweep, such as in the range of 0.001-50 kHz, such as in the range of 0.1-50 kHz, especially in the range of 1-30 kHz. The term “frequency sweep” may herein especially refer to starting at a first frequency and continuously or step-wise adjusting the frequency until a terminal frequency, and optionally (continuously or step-wise) reverting to the starting frequency.

The embodiments described herein are not limited to a single aspect of the invention. For example, an embodiment describing the method may, for example, further relate to the system, especially to an operational mode of the system, or especially to the control system. Similarly, an embodiment of the system describing an operation of the system may further relate to embodiments of the method. In particular, an embodiment of the method describing an operation (of the system) may indicate that the system may, in embodiments, be configured for and/or be suitable for the operation. Similarly, an embodiment of the system describing actions of (a stage in) an operational mode may indicate that the method may, in embodiments, comprise those actions.

### BRIEF DESCRIPTION OF THE DRAWINGS

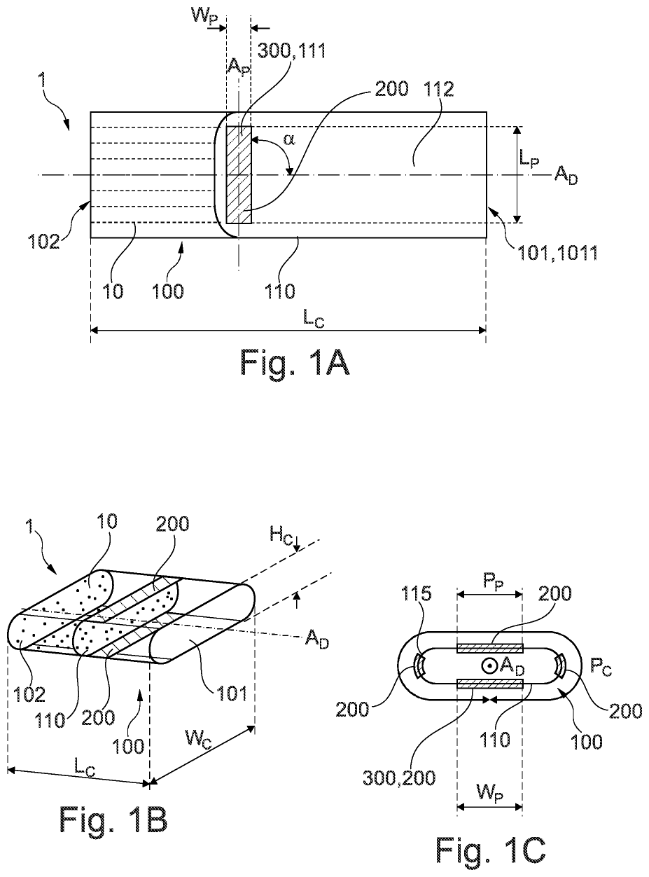

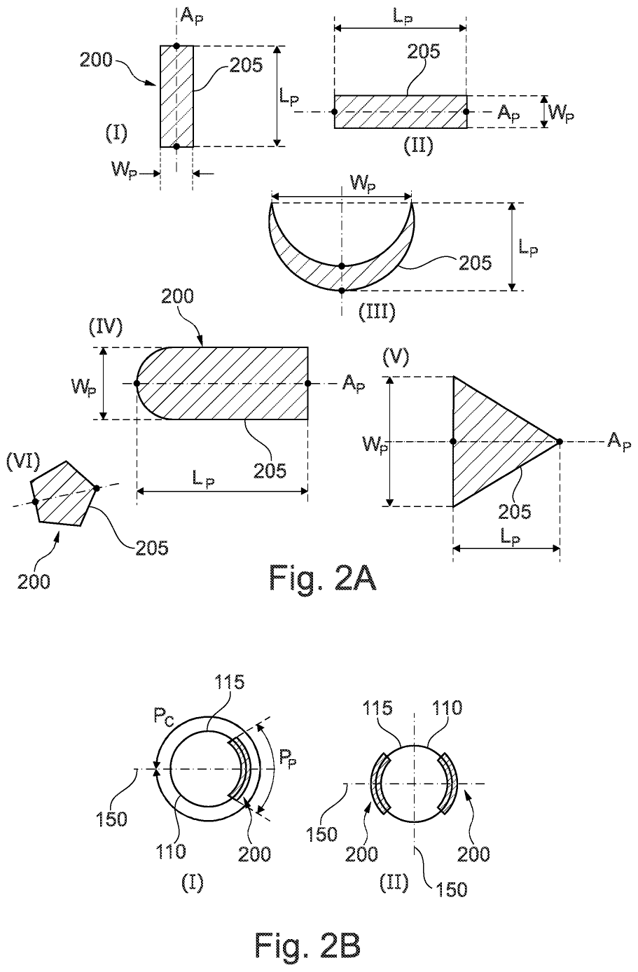

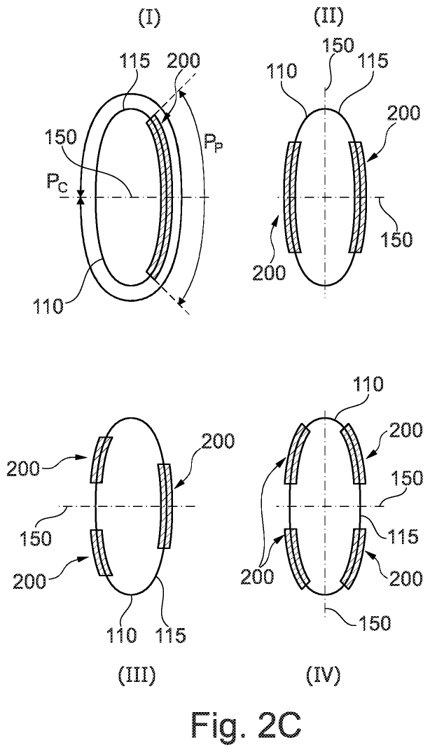

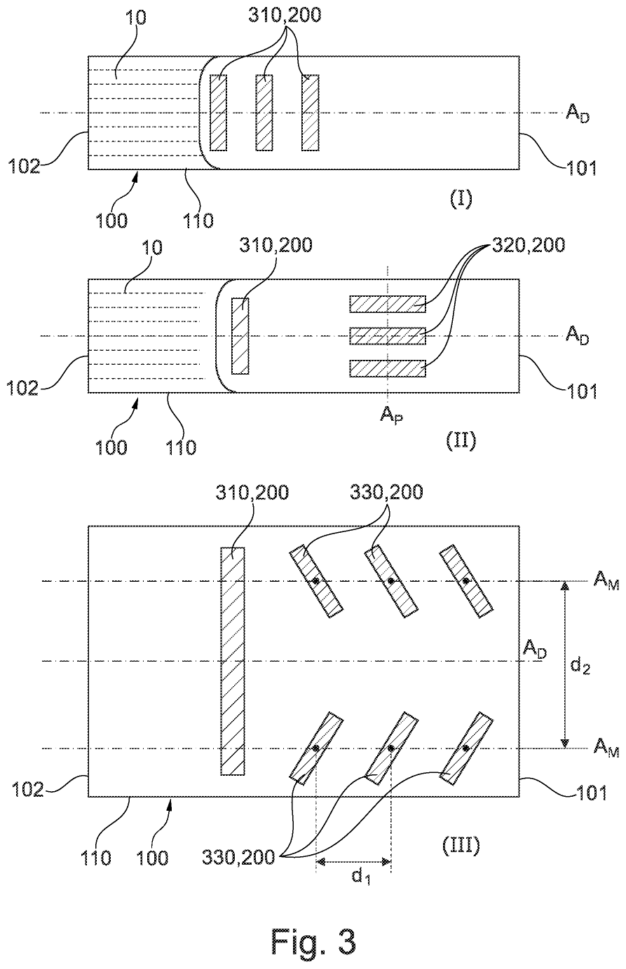

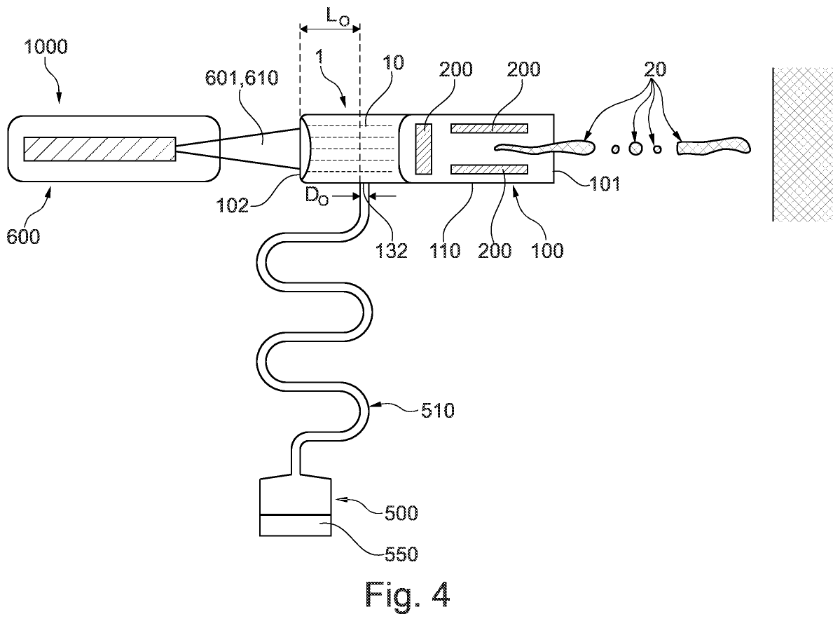

Embodiments of the invention will now be described, by way of example only, with reference to the accompanying schematic drawings in which corresponding reference symbols indicate corresponding parts, and in which: FIG. 1A-C schematically depict embodiments of the microfluidic device 1, FIG. 2A schematically depicts patches 200 of different shapes in embodiments, FIG. 2B and FIG. 2C schematically depict the plane of symmetry 150 of the pattern 300 in embodiments, FIG. 3 schematically depicts the pattern 300 comprising a plurality of sets of patches 200 in embodiments, and FIG. 4 schematically depicts an embodiment of a jet ejection system 1000. The schematic drawings are not necessarily on scale.

### DETAILED DESCRIPTION OF THE EMBODIMENTS

FIG. 1A-C schematically depict embodiments of the microfluidic device 1. FIG. 1A shows a cross-section of the microfluidic device 1 in a cross-sectional plane parallel to the device axis of elongation A_{D}. FIG. 1B depicts an isometric view of a different embodiment of the microfluidic device 1, and FIG. 1C depicts a cross-section of yet another embodiment of the microfluidic device 1 in a cross-sectional plane perpendicular to the device axis of elongation A_{D}.

In embodiments, the invention may provide a microfluidic device 1 for jet ejection. In embodiments, the microfluidic device 1 may comprise a hosting chamber 100 defined by a chamber wall 110. Especially, the hosting chamber 100 may be configured to host a liquid 10. In embodiments, along a device axis of elongation A_{D} the hosting chamber 100 may have a chamber length L_{C} defined by a first chamber end 101 and a second chamber end 102. Note that, in embodiments, the chamber wall 110 may comprise the first chamber end 101 and the second chamber end 102. Especially, the first chamber end 101 may comprise a first chamber opening 1011 for jet ejection from the hosting chamber 100. In the embodiments depicted i.e., in FIG. 1A-C, the first chamber opening 1011 is as large as the first chamber end 101. However, in other embodiments, the first chamber opening 1011 may (only) be a portion of the of the first chamber end 101.

Especially, in embodiments, the first chamber end may be (fully) open. In particular, the hosting chamber 100 may have an average area S_{H} in cross-sections perpendicular to the device axis of elongation A_{D}, and the first chamber opening may (in a plane perpendicular to the device axis of elongation A_{D}) have an opening area selected from the range of 0.5*S_{H}-S_{H}, especially from the range of 0.9*S_{H}-S_{H}, such as (essentially) S_{H}.

In embodiments, the chamber wall 110 may comprise a (surface) pattern 300 of a first (repelling) surface material 111 and a second (affinity) surface material 112. Especially, the first surface material 111 may have an equilibrium contact angle θ₁>90° for the liquid 10, and the second surface material 112 may have an equilibrium contact angle θ₂ for the liquid 10. More especially, θ₁−θ₂≥20°.

In embodiments, the (surface) pattern 300 may comprise a (surface) patch 200. In embodiments, the patch 200 may be a two-dimensional shape defined on the surface of the chamber wall 110. In embodiments, the patch may have a patch boundary 205 (see FIG. 2A). Further, in embodiments, the patch 200 may comprise one of the first surface material 111 and the second surface material 112. Further, in embodiments, at least 50% of the patch boundary 205 may contact the other of the first surface material 111 and the second surface material 112. In embodiments, the patch 200 may comprise the first surface material 111 and at least 80% of the patch boundary 205 may contact the second surface material 112. Additionally, in embodiments, the patch 200 may comprise the second surface material 112 and at least 80% of the patch boundary 205 may contact the first surface material 111. In further embodiments, the patch may contact the other of the first surface material 111 and the second surface material 112 along ≥99% of the patch boundary 205. Generally, the patch 200 may in embodiments be configured away from the first chamber end 101 (such as in the embodiments depicted in FIGS. 1A and 1B). In such embodiments, the patch 200 may be completely surrounded by the other of the first surface material 111 and the second surface material 112. Hence, in such embodiments at least 99% of the patch boundary 205, or (even) 100% of the patch boundary 205 may contact the other of the first surface material 111 and the second surface material 112. However, in other embodiments, the patch 200 may be configured such that patch boundary 205 may be coincident with the first chamber opening 1011. In such embodiments, only a part of the patch 200 may be surrounded by the other of the first surface material 111 and the second surface material 112. Therefore, in such embodiments, at least 50%, such as at least 60%, especially at least 70%, more especially at least 80% of the patch boundary 205 may contact the other of the first surface material 111 and the second surface material 112.

Note that, in embodiments, the pattern 300 may (also) comprise a plurality of patches 200. Only one patch 200 can be observed in the embodiment shown in FIG. 1A. The embodiments depicted in FIGS. 1B and 1C depict two and four patches 200, respectively.

In embodiments, the chamber wall 110 may have a (wall) surface area S_{W}. Especially, the (surface) patch 200 has a (patch) surface area S_{P}. More especially, 10⁻⁴≤S_{P}/S_{W}≤2*10⁻¹.

In embodiments, the patch 200 may have a patch axis of elongation A_{P}. Especially, the patch 200 may have a patch length L_{P} (see also FIG. 2A) along the patch axis of elongation A_{P} and a patch width W_{P} perpendicular to the patch axis of elongation A_{P}. In embodiments, the patch width W_{P} may be measured along the chamber wall 110. Especially, L_{P}≥1.5*W_{P}. In the embodiments depicted in FIGS. 1A and 1B, the patch axis of elongation A_{P} may be perpendicular to the device axis of elongation A_{D}.

In embodiments, the patch 200 may have a patch shape approximating a shape selected from the group comprising a triangle, a trapezoid, especially a rectangle, a crescent moon, an oval, a circle, etc. In embodiments, the patch shape may approximate a triangle. In further embodiments, the patch shape may approximate a trapezoid. Yet further, in embodiments, the patch shape may approximate a rectangle. In other embodiments, the patch shape may approximate a crescent. Especially, the patch shape may approximate an oval. More especially, the patch shape may approximate a circle.

In further embodiments, the patch shape may be similar to the aforementioned shapes, however the curvature of one or more sides of the aforementioned shapes may be different. For example, the patch shape may be triangular-like, wherein the patch shape may be a triangle with one curved side. Hence, in this way, the patch shape may approximate a shape selected from the group comprising a triangle, a trapezoid, especially a rectangle, a crescent moon, an oval, a circle, etc. In embodiments, the patch 200 may have an outline defined by a plurality of line segments, wherein at most two of the line segments are curved. A plurality of different patch shapes in embodiments are depicted in FIG. 2A.

The term “approximate” and its conjugations herein, such as in “to approximate a shape”, refers to being nearly identical to, especially identical to, the following term, for example nearly identical to a circular sector or a semi-cylindrical shape. For example, a patch boundary 205 may define a circular patch 200 but for a defect. Similarly, for example, the rounded shape defined by the patch 200 may not be perfectly round but slightly ellipsoidal. In particular, an object approximating a first shape may herein refer to: a first shape realization encompassing the object, wherein the first shape realization is defined as the smallest encompassing shape of the (2D or 3D, respectively) object wherein the first shape realization has the shape of the first shape, wherein a ratio of the area (volume) of the first shape realization to the area (volume) of the object is ≤1.2, especially ≤1.1, such as ≤1.05, especially ≤1.02. For instance, a patch 200 may approximate a semi-cylindrical shape, wherein the first shape realization may be defined as the smallest encompassing semi-cylindrical shape of the patch 200, wherein a ratio of the volume of the first shape realization to the volume of the patch 200 is ≤1.2, especially ≤1.1, such as ≤1.05, especially ≤1.02, including 1. Further, if the dimensions of the first shape are defined, the term approximate may refer to the object and the first shape being superimposable (in 2D or 3D, respectively) such that an intersection between the object and the first shape covers at least n % of the object and at least n % of the shape, wherein n is at least 90%, such as at least 95%, especially at least 98%, such as at least 99%, including 100%.

In embodiments, the patch 200 may comprise the first surface material 111. Especially, the patch axis of elongation A_{P} may make an angle α≥80° with the device axis of elongation A_{D}. In embodiments, the patch width W_{P} may be selected from the range of 0.01*L_{C}-0.2*L_{C}.

In embodiments, the patch 200 may comprise the second surface material 112. Especially, the patch axis of elongation A_{P} may make an angle α≤30° with the device axis of elongation A_{D}. In embodiments, the patch length L_{P} may be selected from the range of 0.2*L_{C}-0.95*L_{C}.

Note that, the angle α may especially be the smallest angle between the device axis of elongation (A_{D}) and the patch axis of elongation (A_{P}) i.e., no distinction is made between −α and +α, both may be considered α.

In embodiments, it may apply that the pattern 300 (of the first surface material 111 and the second surface material 112) may have a plane of symmetry 150 (see FIG. 2B). Especially, the device axis of elongation A_{D} may be coincident with the plane of symmetry 150, or alternatively (or additionally) the device axis of elongation A_{D} may be perpendicular to the plane of symmetry 150. Some embodiments where patterns have a plane of symmetry 150 are depicted in FIGS. 2B and 2C.

In embodiments, in a cross-section perpendicular to the device axis of elongation A_{D} the hosting chamber 100 may have a perimeter 115 (or “circumference”) with a (chamber) perimeter length P_{C}. Note that the perimeter 115 is indicated in FIG. 1C, wherein the extent of the perimeter 115 of the chamber wall 110 (in a cross-section perpendicular to the device axis of elongation A_{D}) is, for visualizational purposes, schematically marked by an additional (closed) line (with arrows on either end). This has (also) been indicated in other embodiments in a similar manner in FIG. 2B (I) and FIG. 2C (I). Especially, the patch 200 may cover a (patch) perimeter length P_{P} along the perimeter 115. In embodiments, P_{P} may be selected from the range of 0.05*P_{C}-0.25*P_{C}.

In embodiments, in a cross-section perpendicular to the device axis of elongation A_{D} the hosting chamber 100 has a perimeter 115 (or “circumference”) with a (chamber) perimeter length P_{C}. Especially, the patch 200 may cover a (patch) perimeter length P_{P} along the perimeter 115. More especially, P_{P} may be selected from the range of 0.4*P_{C}-P_{C}.

In embodiments, the pattern 300 may comprise a plurality of patches 200. Especially, the pattern 300 may comprise a plurality of sets of patches 200. FIG. 3 depicts such embodiments.

In embodiments, the hosting chamber 100 may have a chamber height H_{C} selected from the range of 5-400 μm, a chamber width W_{C} selected from the range of 2*H_{C}-10*H_{C}, and a chamber length L_{C} selected from the range of 100-5000 μm. Further, in embodiments, along at least 80% of the chamber length L_{C} the hosting chamber 100 may have a cross-sectional shape approximating a shape selected from the group comprising a rounded rectangle, a stadium, and an oval.

FIG. 2A schematically depicts patches 200 of different shapes in embodiments.

In embodiments, the patch 200 may be defined by a plurality of sides. Embodiment I depicts a rectangular patch 200 defined by a longer length L_{P} as compared to the width W_{P}. In embodiments, the patch axis of elongation A_{P} may pass through the midpoints of at least one side of the patch 200.

Furthermore, in embodiments, two sides of the patch 200 may be configured parallel to the patch axis of elongation A_{P}. Note that the orientation of the patch 200 may influence the heterogenous surface chemistry of the chamber wall 110. Especially, the patch 200 may be configured at an angle α with the device angle of elongation A_{D}. Embodiment II is similar to embodiment I, however the patch 200 in embodiment II is oriented in a perpendicular direction to embodiment I.

In embodiment III of FIG. 2A, the patch 200 comprises a crescent shaped patch 200. Here, the patch 200 may especially be defined by just two curves of different radii forming a crescent shape. Note that in such embodiments, (alternative to a patch axis of elongation A_{P}) a patch axis of orientation A_{O} may be defined especially passing through the midpoint of the two curves (indicated by the dashed line).

In embodiment IV of FIG. 2A, the patch 200 comprises a half-stadium shape. A half-stadium shape may be defined by two parallel and equal sides and two connecting lines that connect the ends of the parallel sides to form a close two-dimensional region, wherein one of the two connecting sides is a straight line and the other is a curved line. In this embodiment, the patch length L_{P} of the patch 200 may be measured along the direction of the parallel sides and the patch width W_{P} may be measured as the distance between the two parallel sides (as indicated in the figure).

The patch axis of elongation A_{P} of a (2D) shape may herein especially refer to an axis oriented along the direction of elongation and passing through the fictional center of mass of the (2D) shape (would the (2D) shape have an arbitrary thickness). In embodiments, the axis of elongation A_{P} may pass through the midpoints of the shortest sides of the smallest rectangle realization encompassing the shape.

In embodiment V of FIG. 2A, the patch 200 comprises a patch 200 comprising three sides. In such an embodiment, the patch axis of elongation A_{P} may be defined passing through the midpoint of one of the sides and through the point of intersection of the other two sides. In embodiments, wherein the patch 200 may comprise an odd number of sides, the patch axis of elongation A_{P} may be defined through the midpoint of one of the sides and through the intersection of two other sides opposite to said side (such as depicted in embodiment VI).

Note that in embodiments, the patch 200 may (also) have other patch shapes approximating a shape selected from the group comprising a triangle, a trapezoid, especially a rectangle, a crescent moon, an oval, a circle, etc. Furthermore, in embodiments, the patch 200 may have a wave-like shape, wherein the patch length L_{P} in such an embodiment may be measured in a direction along the same direction in which the wavelength may be measured and the patch width W_{P} may be measured as the distance between the crest and the trough of the wave-like shape. In further embodiments, the patch 200 may have an outline defined by a plurality of line segments. Especially, at most two of the line segments may be curved.

FIG. 2B and FIG. 2C schematically depict embodiments illustrating the plane of symmetry 150 of the pattern 300. In embodiments, in a cross-section perpendicular to the device axis of elongation A_{D} the hosting chamber 100 may have a perimeter 115 (or “circumference”) with a (chamber) perimeter length P_{C}. Especially, the patch 200 may cover a (patch) perimeter length P_{P} along the perimeter 115. In embodiments, P_{P} may be selected from the range of 0.05*P_{C}-0.25*P_{C}. In further embodiments, P_{P} may be selected from the range of 0.4*P_{C}-P_{C}.

FIG. 2B depicts a cross-section perpendicular to the device axis of elongation A_{D} of embodiments comprising a hosting chamber 100 with a circular (or “rounded”) cross-section. Embodiment I comprises a pattern 300 comprising (only) one patch 200. In the depicted embodiment, the pattern 300 may have one plane of symmetry 150. Likewise, in embodiment II, the pattern 300 comprises two patches 200. In the depicted embodiment, the pattern 300 may have two planes of symmetry 150. The planes of symmetry 150 are indicated by dashed lines. In the depicted embodiments, the pattern 300 may have a further plane of symmetry 150, such as the cross-sectional plane.

FIG. 2C depicts a cross-section perpendicular to the device axis of elongation A_{D} of embodiments comprising a hosting chamber 100 with an oval cross-section. Here, the chamber wall 110 in embodiment I, II, III and IV comprises one, two, three and four patches 200, respectively. Analogous to the embodiments in FIG. 2B, the pattern 300 may in embodiments comprise at least one plane of symmetry 150. For instance, the pattern 300 in embodiments I and III comprise (at least) one plane of symmetry 150. The pattern 300 in embodiments II and IV have (at least) two planes of symmetry.

FIG. 3 schematically depicts embodiments wherein the pattern 300 comprises a plurality of sets of patches 200.

In embodiment I, the pattern 300 comprises a first set 310 of n patches 200. Especially, n may at least be two. In embodiment I n is three. In embodiments, the n patches 200 may be arranged successively (and especially equidistantly) (in a direction parallel to the device axis of elongation A_{D}) downstream from the second chamber end 102. Further, in embodiments, each patch 200 of the first set 310 may comprise the first surface material 111. Yet further, in embodiments, the patch axis of elongation A_{P} of each patch 200 of the first set 310 may make an angle α≥80° with the device axis of elongation A_{D}. In the embodiment depicted, the liquid is filled up to the first patch 200 of the first set 310 of n patches 200.

In embodiment II, the pattern 300 comprises a second set 320 of k patches 200. Especially, k may at least be two. In embodiment II, k is three. Note that, in embodiments, the pattern 300 may have a plurality of sets of patches 200. For instance, in the embodiment depicted, the pattern 300 comprises a first set 310 of one patch 200 and the second set 320 of three patches 200.

Further, in embodiments, each patch 200 of the second set 320 may comprise the second surface material 112. Especially, each patch 200 of the second set 320 may be configured at a (shortest) distance selected from the range of 0.3*L_{C}-0.9*L_{C} from the second chamber end 102 (measured along the device axis of elongation A_{D}). In embodiments, the patch axis of elongation A_{P} of each patch 200 of the second set 320 may make an angle α≤30° with the device axis of elongation A_{D}. Additionally or alternatively, in embodiments, the patches 200 of the second set 320 may converge or diverge towards the first chamber end 101.

As mentioned before, the pattern 300 may comprise a plurality of sets, wherein each set may comprise a plurality of patches 200. Embodiment III depicts a microfluidic device comprising a plurality of third sets 330.

In embodiments, each third set 330 may comprise a set of m patches 200. In embodiments, m may at least be two. Embodiment III comprises two third sets 330 of m patches 200, wherein each third set 330 comprises three patches 200.

In embodiments, each patch 200 of the third set 330 may comprise the second surface material 112. Especially, the centroid of (all) the patches 200 may lie along an axis A_{M} defined on the chamber wall 110. More especially, the axis A_{M} may be parallel to the device axis of elongation A_{D}. In embodiments, the patch axis of elongation A_{P} of the m patches 200 of the third set 330 may make an angle 5°≤α≤50° with the device axis of elongation A_{D}. Further, in embodiments, the centroids of two consecutive patches 200 of the third set 330 may be configured at a first distance d1. Especially, d1 may be selected from the range of 0.05*L_{C}-0.5*L_{C}.

Furthermore, in embodiments, the axes A_{M} of two neighboring third sets 330 may be arranged at a distance d2. Especially, d2 (measured in a perpendicular direction to the axis A_{M} and along the chamber wall 110) may be selected from the range of 0.2*P_{C}-0.5*P_{C}. In the embodiment depicted in FIG. 3 (III), the first third set 330 makes a positive angle α and the second third set 330 makes a negative angle α. Hence, in this way, (patches 200 of) the plurality of third sets 330 may converge (or diverge) towards the first chamber end 101. In particular, in the embodiment depicted in FIG. 3 (III), the patches 200 of the plurality of third sets 330 may converge towards the first chamber end 101, especially the patches 200 of different third sets 330 may converge towards the first chamber end 101.

FIG. 4 schematically depicts an embodiment of a jet ejection system 1000.

In a further aspect, the invention may provide a jet ejection system 1000 comprising (i) the microfluidic device 1, (ii) a liquid supply 500 and (iii) a (laser-based) heating system 600. Note that the heating system 600 may in embodiments facilitate providing energy to the liquid 10 hosted in the hosting chamber 100. It will be apparent to the skilled person that other sources of energy may in embodiments (also) be used to provide energy to the liquid 10, for example by means of a piston, Joule heating, dielectric breakdown, etc. Some of these embodiments have been discussed further above.

In embodiments, the hosting chamber 100 may comprise a hosting chamber opening 132. Further, in embodiments, the liquid supply 500 (comprising stored liquid 550) may be configured to provide the liquid 10 to the hosting chamber 100 via the hosting chamber opening 132. Especially, tubes or (micro) pipes 510 may be used to connect the liquid supply 500 to the hosting chamber opening 132. Furthermore, the (laser-based) heating system 600 may be configured to provide (laser) radiation 601 to one or more of the chamber wall 110 and the liquid 10 in the hosting chamber 100. Especially, in embodiments, the laser radiation 601 may comprise an infrared laser pulse 610.

In embodiments, the hosting chamber opening 132 may be located at a distance L_{O} from the second chamber end 102. In embodiments, L_{O} may be selected from the range of 10-2000 μm. Note that in some embodiments, the hosting chamber opening 132 may (also) be configured in the second chamber end 102, i.e. L_{O}=0. Further, in embodiments, the hosting chamber opening 132 may have a diameter D_{O}. Especially, D_{O} may be selected from the range of 1-1000 μm.

In the embodiment depicted in FIG. 4, the (laser-based) heating system 600 may (be configured to) shine a beam of laser radiation 601 onto the liquid 10 via the chamber wall 110. The energy provided by the laser radiation 601 may especially be absorbed by the liquid 10 in the hosting chamber 100.

Further, in embodiments, the liquid 10 may in embodiments be filled to a predetermined volume (via the hosting chamber opening 132), wherein the advancement of the liquid meniscus may be arrested by means of the patch 200.

The heat provided by the laser radiation 601 may vaporize at least a part of the liquid 10. The expansion of the vaporized liquid 10 may accelerate the liquid 10 along the device axis of elongation A_{D}. Thus, the microfluidic jet 20 may be ejected from the microfluidic device 1.

In a further aspect, the invention provides a method for ejecting a jet 20 with the microfluidic device 1. In embodiments, the method may comprise a liquid provision step and an ejection step. In embodiments, the liquid provision step may comprise providing the liquid 10 to the hosting chamber 100. Especially, the liquid provision step may comprise filling 20-70 vol. % of the hosting chamber 100 with the liquid 10.

In embodiments, the ejection step may comprise providing radiation 601 to the chamber wall 110 and/or to the liquid 10 such that at least part of the liquid 10 is boiled (or vaporized) and a liquid jet 20 is ejected. Further, in embodiments, the ejection step may comprise vaporizing part of the liquid 10 (in the hosting chamber 100) by heating the liquid 10. Especially, heat may be provided at a power selected from the range of 0.1-10 W.

The term “plurality” refers to two or more. Furthermore, the terms “a plurality of” and “a number of” may be used interchangeably. The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A microfluidic device (1) for jet ejection, wherein the microfluidic device (1) comprises a hosting chamber (100) defined by a chamber wall (110), wherein:
the hosting chamber (100) is configured to host a liquid (10), wherein along a device axis of elongation (A_{D}) the hosting chamber (100) has a chamber length (L_{C}) defined by a first chamber end (101) and a second chamber end (102), wherein the first chamber end (101) comprises a first chamber opening (1011) for jet ejection from the hosting chamber (100);
the chamber wall (110) comprises a pattern (300) of a first surface material (111) and a second surface material (112), wherein the first surface material (111) has an equilibrium contact angle θ₁>90° for the liquid (10), and wherein the second surface material (112) has an equilibrium contact angle θ₂ for the liquid (10), wherein θ₁−θ₂≥20°;
the pattern (300) comprises a patch (200), wherein the patch has a patch boundary (205), and wherein (a) the patch (200) comprises one of the first surface material (111) and the second surface material (112), and wherein (b) at least 50% of the patch boundary (205) contacts the other of the first surface material (111) and the second surface material (112);
the chamber wall (110) has a wall surface area (S_{W}), wherein the patch (200) has a patch surface area (S_{P}), wherein 10⁻⁴≤S_{P}/S_{W}≤2*10⁻¹; and
in a cross-section perpendicular to the device axis of elongation (A_{D}) the hosting chamber (100) has a perimeter (115) with a chamber perimeter length P_{C}, wherein the patch (200) covers a patch perimeter length P_{P} along the perimeter (115), wherein P_{P} is selected from the range of 0.01*P_{C}-0.5*P_{C}.

2. The microfluidic device (1) according to claim 1, wherein the patch (200) has a patch axis of elongation (A_{P}), wherein the patch (200) has a patch length (L_{P}) along the patch axis of elongation (A_{P}) and a patch width (W_{P}) perpendicular to the patch axis of elongation (A_{P}), wherein L_{P}≥1.5*W_{P}.

3. The microfluidic device (1) according to claim 2, wherein the patch (200) comprises the first surface material (111), wherein the patch axis of elongation (A_{P}) makes an angle α≥80° with the device axis of elongation (A_{D}), and wherein the patch width W_{P} is selected from the range of 0.01*L_{C}-0.2*L_{C}.

4. The microfluidic device (1) according to claim 2, wherein the patch (200) comprises the second surface material (112), wherein the patch axis of elongation (A_{P}) makes an angle α≤30° with the device axis of elongation (A_{D}), and wherein the patch length (L_{P}) is selected from the range of 0.2*L_{C}-0.95*L_{C}.

5. The microfluidic device (1) according to claim 1, wherein applies that the pattern (300) has a plane of symmetry (150).

6. The microfluidic device (1) according to claim 1, wherein P_{P} is selected from the range of 0.05*P_{C}-0.25*P_{C}.

7. The microfluidic device (1) according to claim 1, wherein the patch contacts the other of the first surface material (111) and the second surface material (112) along ≥99% of the patch boundary, and wherein the patch (200) has an outline defined by a plurality of line segments, wherein at most two of the line segments are curved.

8. The microfluidic device (1) according to claim 2, wherein the pattern (300) comprises a first set (310) of n patches (200), wherein n≥2, wherein:
the n patches (200) are arranged successively downstream from the second chamber end (102);
each patch (200) of the first set (310) comprises the first surface material (111);
the patch axis of elongation (A_{P}) of each patch (200) of the first set (310) makes an angle α≥80° with the device axis of elongation (A_{D}).

9. The microfluidic device (1) according to claim 2, wherein the pattern (300) comprises a second set (320) of k patches (200), wherein k≥2, wherein:
each patch (200) of the second set (320) comprises the second surface material (112);
each patch (200) of the second set (320) is configured at a distance selected from the range of 0.3*L_{C}-0.9*L_{C} from the second chamber end (102);
the patch axis of elongation (A_{P}) of each patch (200) of the second set (320) makes an angle α≤30° with the device axis of elongation (A_{D}).

10. The microfluidic device (1) according to claim 9, wherein k=2, and wherein the patches (200) of the second set (320) converge or diverge towards the first chamber end (101).

11. The microfluidic device (1) according to claim 2, wherein the pattern (300) comprises a plurality of third sets (330), wherein:
each third set (330) comprises a set of m patches (200), wherein m≥2, wherein each patch (200) of the third set (330) comprises the second surface material (112), wherein the centroid of the patches (200) lie along an axis (A_{M}) defined on the chamber wall (110), wherein the axis (A_{M}) is parallel to the device axis of elongation (A_{D}), wherein the patch axis of elongation (A_{P}) of the m patches (200) of the third set (330) makes an angle 5°≤α≤50° with the device axis of elongation (A_{D}), wherein the centroids of two consecutive patches (200) of the third set (330) are configured at a first distance d1, wherein d1 is selected from the range of 0.05*L_{C}-0.5*L_{C}.

12. The microfluidic device (1) according to claim 11, wherein the axes (A_{M}) of two neighboring third sets (330) are arranged at a distance d2, wherein d2 is selected from the range of 0.2*P_{C}-0.5*P_{C}.

13. The microfluidic device (1) according to claim 1, wherein the hosting chamber (100) has a chamber height (H_{C}) selected from the range of 5-400 μm, a chamber width (W_{C}) selected from the range of 2*H_{C}-10*H_{C}, and a chamber length (L_{C}) selected from the range of 100-5000 μm, and wherein along at least 80% of the chamber length (L_{C}) the hosting chamber (100) has a cross-sectional shape approximating a shape selected from the group comprising a rounded rectangle, a stadium, and an oval.

14. A jet ejection system (1000) comprising (i) the microfluidic device (1) according to claim 1, (ii) a liquid supply (500) and (iii) a heating system (600), wherein:
the hosting chamber (100) comprises a hosting chamber opening (132);
the liquid supply (500) is configured to provide the liquid (10) to the hosting chamber via the hosting chamber opening (132);
the heating system (600) is configured to provide radiation (601) to one or more of the chamber wall (110) and the liquid (10) in the hosting chamber (100).

15. A method for ejecting a jet (20) with the microfluidic device (1) according to claim 1, wherein the method comprises:
a liquid provision step comprising providing the liquid (10) to the hosting chamber (100), wherein the liquid provision step comprises filling 20-70 vol. % of the hosting chamber (100) with the liquid (10);
an ejection step comprising providing radiation (601) to the chamber wall (110) and/or to the liquid (10) such that at least part of the liquid (10) is boiled and a liquid jet (20) is ejected.
